(19) United States
(12) Patent Application Publication
(10) Pub. No.: US 2015/0258022 A1
(43) Pub. Date: Sep. 17, 2015

(54) Formulation for the Delivery of Nucleotide Sequences That Can Modulate Endogenous Interfering RNA Mechanisms

(71) Applicant: Commissariat à I'énergie atomique et aux énergies alternatives, PARIS (FR)

(72) Inventors: Fabrice Navarro Y Garcia, Fontaine (FR); Jonathan Bruniaux, Grenoble (FR); Xavier Gidrol, Antony (FR); Eric Sulpice, Meylan (FR); Isabelle Texier-Nogues, Grenoble (FR)

(21) Appl. No.: 14/424,623

(22) PCT Filed: Aug. 12, 2013
(86) PCT No.: PCT/EP2013/066821

§ 371 Date: Feb. 27, 2015

(30) Foreign Application Priority Data
Aug. 30, 2012 (FR) 12 58115

(51) Int. Cl. A61K 9/107; A61K 47/12; A61K 47/10; A61K 49/00; A61K 31/713

(52) U.S. Cl. CPC A61K 9/1075; A61K 49/0004; A61K 31/713; A61K 47/10; A61K 47/12

(57) ABSTRACT

The present invention concerns a formulation in nanoemulsion form comprising a continuous aqueous phase and at least one dispersed phase, useful as system for delivering nucleotide sequences able to modulate endogenous mechanisms of RNA interference or as transfecting agent.

The present invention concerns a formulation of nanoemulsion type useful for delivering nucleotide sequences capable of modulating endogenous mechanisms of interfering RNA.

The mechanism of RNA interference is a mechanism of post-transcriptional inhibition of gene expression and is based on double-strand small interfering RNAs specifically degrading a target mRNA and inhibiting the expression of a protein highly selectively and specifically. This mechanism takes place in the cytoplasm and involves a multi-protein complex called the RISC complex (RNA-induced silencing complex).

Since the discovery of this natural mechanism, nucleotide sequences able to modulate endogenous mechanisms of interfering RNAs have been used in numerous academic and/or pharmaceutical laboratories as research tools in several biology sectors. For example the nucleotide sequences able to modulate endogenous mechanisms of interfering RNAs are used to detect new therapeutic targets in numerous pathologies. Through the highly selective and specific inhibition of the expression of a given protein it is possible to evidence the role it plays in the development process of the pathology. Nevertheless the inserting of exogenous genetic material into a cell (transfection) requires an adapted physical, chemical or biological method to allow these nucleotide sequences able to modulate endogenous mechanisms of RNA interference—which are generally large molecules (>10 kDa) and negatively charged—to pass through the negatively charged plasma membrane which forms a veritable barrier.

Among the physical methods mention can be made of electroporation or the generating of pores in the membrane via ultrasound. Among the biological methods, recourse can be had to systems specialised in delivering nucleic acids such as viruses. Among the chemical methods the use can be cited of:
lipoplexes (complexes formed between nucleic acids and cationic lipids) e.g. Lipofectamine® 2000,
polyplexes (complexes formed between nucleic acids and cationic polymers e.g. polyethyleneimine PEI),
complexes formed between nucleic acids and dendrimers e.g. dendrimers containing poly(amidoamine) PANAM,
lipopolyplexes (complexes formed between nucleic acids, liposomes and polymers) e.g. SNALPs (<<stable nucleic acid-lipid particles>>) which correspond to mixtures of cationic lipids and auxiliaries forming liposomes stabilised by polymers of polyethylene-glycol (PEG) type;
complexes formed between nucleic acids and polymeric poly(DL-lactide-co-glycolide) PLGA nanoparticles.

To the knowledge of the inventors, solely Park et al. (Molecular Pharmaceutics 5, 4, 622-631, 2008) describe the use of lipid nanoparticles to deliver siRNA. The lipid nanoparticles comprise cholesterol, 1,2-Dioleoyl-sn-Glycero-3-Phosphoethanolamine (DOPE) and a siRNA derivative and were used for the transfection and knockdown of the GFP protein. However to prevent leakage of siRNA outside the nanoparticles, the above were covalently grafted thereto. siRNAs were covalently bonded to a poly(ethylene oxide) chain intercalated in the nanoparticles and allowed the fixing of the siRNAs. In addition, a fluorescent marker (imaging agent) was also covalently grafted to the siRNAs to track transfection. However, any chemical modification of siRNA is likely to cause denaturing thereof. Additionally the technique used by Park et al. requires the chemical modification of each siRNA used, which is time-consuming and costly. There is therefore a need to provide a system for delivering nucleotide sequences capable of modulating endogenous mechanisms of RNA interference wherein these sequences are not chemically modified.

Moreover, patent application WO 2010/018223 describes the use of a formulation in the form of a nanoemulsion comprising an aqueous phase and at least one dispersed phase comprising an amphiphilic lipid a solubilising lipid, a therapeutic agent and a co-surfactant comprising at least one chain composed of alkylene oxide repeat units to deliver an amphiphilic or lipophilic therapeutic agent. The therapeutic agent may notably be a siRNA. However since siRNAs are hydrophilic compounds they would be contained in the continuous aqueous phase of the nanoemulsion and not in the dispersed phase. The formulation described in this application is therefore not adapted for the delivering of nucleotide sequences able to modulate endogenous mechanisms of interfering RNA. In addition, the present inventors have evidenced that all the formulations described in WO 2010/018223, and in particular those described in the examples, even when integrating cationic surfactants therein, are not adapted for the present application since the yield of complexing is very low, and transfection would not be efficient. As explained below, only formulations comprising specific proportions of components allow the delivering of nucleotide sequences capable of modulating endogenous mechanisms of interfering RNA.

Also systems for delivering deoxyribonucleic acid (DNA) containing an oil-in-water emulsion or lipid droplets are known. In these systems the lipid droplets comprise cationic surfactants via which the DNA binds to the droplets through electrostatic interactions.

For example, Del Pozo-Rodriguez et al. (International Journal of Pharmaceutics, 385, 2010, 157-162) observed the presence of fluorescence in the liver and spleen of mice which were intravenously given lipid nanoparticles comprising naked plasmid DNA (pDNA) expressing the fluorescent protein EGFP, Precirol® ATO 5, Tween 80, and 1,2-dioleoyl-3-trimethylammoniumpropane chloride (DOTAP) (cationic surfactant) for in vitro transfection of HEK293 cells and in vivo after administering to mice.

Tabatt et al. (European Journal of Pharmaceutics and Biopharmaceutics 57, 2004, 155) describe the use of solid lipid nanoparticles comprising Compritol ATO 888 or cetylpalmitate, Tween 80, Span 85, a cationic surfactant selected from among alkyldimethylbenzylammonium chloride (BA), tetradecyltrimethylammonium bromide (CTAB), hexadecylpyridinium chloride (CPC), dimethyldioctadecylammonium bromide (DDAB), N,N-di-(b-stearoylethyl)-N,N-dimethyl-ammonium chloride (Esterquat 1, EQ 1) and 8, N-[1-(2,3-dioleoyloxy)propyl]-N,N,Ntrimethylammonium chloride (DOTAP) for the in vitro transfection of COS-1 cells.

In both these articles Tween 80 was used. Yet this has some toxicity for the cells. In addition Tween 80 comprises poly(ethylene oxide) chains comprising 20 units of ethylene oxide. As explained below, the inventors have evidenced that a surfactant having longer poly(ethylene oxide) chains (at least 25 ethylene oxide units) is easier to formulate, is more stable and allows more efficient protection of the nucleotide sequences contained in the formulation against the outside medium.

Finally, the nucleotide sequences capable of modulating endogenous mechanisms of RNA interference, in particular siRNA, are generally less stable than DNA. In addition, the biological activities of DNA and siRNA are very different, which means that the delivery systems adapted for DNA are not necessarily the same as those adapted for delivering siRNA. In particular, even if in either case one prerequisite is a delivery system allowing efficient transfection, this is not sufficient for a system to deliver siRNA which will only be of advantage if it allows silencing of the gene.

The development of new formulations allowing in vivo and in vitro delivery of nucleotide sequences capable of modulating endogenous mechanisms of RNA interference is therefore needed.

### [Formulation]

For this purpose and according to a first subject of the invention there is provided a formulation in the form of a nanoemulsion, comprising a continuous aqueous phase and at least one dispersed phase.

The invention provides a formulation comprising a nucleotide sequence able to modulate endogenous mechanisms of interfering RNA which allows the delivery of said sequences.

It also provides a so-called <<premix>> formulation free of nucleotide sequences able to modulate endogenous mechanisms of RNA interference, which allows the preparation of a formulation comprising a nucleotide sequence able to modulate endogenous mechanisms of RNA interference via the complexing of a nucleotide sequence able to modulate endogenous mechanisms of RNA interference with this <<premix>> formulation. This <<premix>> formulation can therefore advantageously be complexed with a nucleotide sequence able to modulate endogenous mechanisms of RNA interference that is adapted in accordance with the desired use of the final formulation.

### [Formulation of Premix Type]

Therefore a first object of the present invention is to provide a formulation in the form of a nanoemulsion, comprising a continuous aqueous phase and at least one dispersed phase, and comprising:
at least 5 mole % of amphiphilic lipid;
15 to 70 mole % of at least one cationic surfactant comprising:
   at least one lipophilic group selected from among:
      an R or R—(C═O)—, group where R is a linear hydrocarbon chain having 11 to 23 carbon atoms,
      an ester or amide of fatty acids having 12 to 24 carbons atoms and phosphatidylethanolamine, and
      a poly(propylene oxide), and
   at least one hydrophilic group comprising at least one cationic group selected from among:
      a linear-chain or branched alkyl group having 1 to 12 carbon atoms and interrupted and/or substituted by at least one cationic group, and
      a hydrophilic polymeric group comprising at least one cationic group, and
10 to 55 mole % of a co-surfactant comprising at least one poly(ethylene oxide) chain having at least 25 units of ethylene oxide;
a solubilising lipid comprising at least one fatty acid glyceride;
optionally a helper lipid;
   where the mole percentages of amphiphilic lipid, cationic surfactant and co-surfactant relate to the whole (amphiphilic lipid/cationic surfactant/co-surfactant/optional helper lipid).

As explained below, the: amphiphilic lipid/cationic surfactant/co-surfactant/optional helper lipid are the chief components in the crown part of the droplet structure in the <<premix>> formulation.

The emulsion is therefore an emulsion of oil-in-water type. It may be simple or multiple in particular comprising a second aqueous phase in the dispersed phase. Preferably it is simple.

The invention is based on the unexpected discovery that the above-described formulation can be used as transfection agent and/or for the in vitro and in vivo delivery of nucleotide sequences able to modulate endogenous mechanisms of RNA interference. The formulation advantageously exhibits good bioavailability and can allow limited degradation of the nucleotide sequences able to modulate endogenous mechanisms of RNA interference that is generally observed with other delivery systems, in particular degradation by proteins.

In addition, the formulation is advantageously stable; it can be stored for several hours without any degradation being observed.

Cationic Surfactant

The formulation of the invention comprises a cationic surfactant comprising:
at least one lipophilic group selected from among:
   an R group representing a linear hydrocarbon chain having 11 to 23 carbon atoms;
   an ester or amide of fatty acids having 12 to 24 carbon atoms and phosphatidylethanolamine, such as distearyl phosphatidylethanolamine (DSPE); and
   a poly(propylene oxide); and
at least one hydrophilic group comprising at least one cationic group selected from among:
   a linear-chain or branched alkyl group having 1 to 12 carbon atoms and interrupted and/or substituted by at least one cationic group; and
   a hydrophilic polymeric group comprising at least one cationic group, the said polymeric group being particularly selected from among:
      a poly(ethylene oxide) typically comprising 3 to 500 ethylene oxide units, preferably 20 to 200 ethylene oxide units, and comprising at least one cationic group,
      a polysaccharide, such as dextran, cellulose or chitosan, particularly having molecular weights of between 0.5 et 20 kDa, for example between 1 and 12 kDa,
      a polyamine, such as a chitosan or polylysine, particularly having molecular weights of between 0.5 and 20 kDa, for example between 1 and 12 kDa.

By <<ester or amide of fatty acids having 12 to 24 carbon atoms and phosphatidylethanolamine>>, is meant a group of formula:

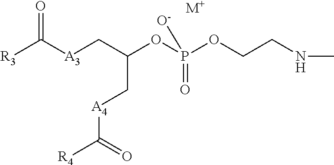

where:
R₃ and R₄ are independently a linear hydrocarbon chain having 11 to 23 carbon atoms;
A₃ and A₄ represent O or NH; and
M is H or a cation.

The cationic groups of the cationic surfactant are typically:
oniums selected from among ammonium, imidazolium, pyridinium, pyrrolidinium, piperidinium, phosphonium or sulfonium groups; or
metal complexes between a radical of a mono- or multi-dentate chelating organic group e.g. phenantroline, pyridine, ethylene diamine tetraacetic acid (EDTA), diethylene triamine pentaacetic acid (DTPA), porphyrins, phtalocyanines, chlorins, bacteriochlorins complexed with an inorganic cation such as Ca²⁺, Al³⁺, Ni⁺, Zn²⁺, Fe²⁺, Fe³⁺ or Cu²⁺,
the ammonium groups, and in particular —⁺NMe₃, —⁺NHMe₂, —⁺NH₂Me and —⁺NH₃, particular preference being given to —⁺NH₃.

Evidently anions are associated with the cationic group(s) so that the formulation is electrically neutral. The type of anion is not limited. As illustrations mention can be made of halides, in particular chlorides or bromides or trifluoroacetate.

In the cationic surfactant the type of linkage group linking the lipophilic group(s) to the hydrophilic group(s) comprising at least one cationic group is not limited. Examples of linkage groups are given below (group L).

In one embodiment, the cationic surfactant has the following formula (A):

[(Lipo)ₗ-L-(Hydro)ₕ]ⁿ⁺,(n/m)[A]^{m-} (A)

where:
l and h are independently integers of between 1 and 4;
n is an integer no lower than 1, generally between 1 and 50;
Lipo represents a lipophilic group such as defined above;
Hydro represents a hydrophilic group such as defined above comprising at least one cationic group;
L is a linkage group;
A is an anion;
m is an integer representing the charge of the anion;
n is an integer representing the charge of the cation [(Lipo)ₗ-L-(Hydro)ₕ].

In above-mentioned formula (A), L is preferably such that:
when l and h represent 1, L is a divalent linkage group selected from among:
   a single bond;
   a Z group selected from among —O—, —NH—, —O(OC)—, —(CO)O—, —(CO)NH—, —NH(CO)—, —O—(CO)—O—, —NH—(CO)—O—, —O—(CO)—NH— and —NH—(CO)—NH, —O—PO(OH)—O— or a cyclic divalent radical having 5 to 6 atoms;
   an Alk group being an alkylene having 1 to 6 carbon atoms; and
   a group: Z-Alk, Alk-Z, Alk-Z-Alk or Z-Alk-Z where Alk and Z are such as defined above and where the two Z groups of the Z-Alk-Z group are the same or different;
when one of the groups l or h represents 1, and the other represents 2, L is a trivalent group selected from among a phosphate group OP—(O—)₃, a group derived from glycerol of formula —O—CH₂—CH—(O—)CH₂—O— and a cyclic trivalent radical having 5 to 6 atoms;
for the other values of l and h, L is a cyclic multivalent radical having 5 to 6 atoms.

In particularly preferred manner L is such that:
when l and h represent 1, L is a divalent linkage group selected from among:
   a single bond;
   a Z group selected from among —O—, —NH—, —O(OC)—, —(CO)O—, —(CO)NH—, —NH(CO)—, —O—(CO)—O—, —NH—(CO)—O—, —O—(CO)—NH— and —NH—(CO)—NH or —O—PO(OH)—O—,
when one of the groups l or h represents 1 and the other represents 2, L is a trivalent group selected from among a phosphate group OP—(O—)₃ and a group derived from glycerol of formula —O—CH₂—CH—(O—)CH₂—O—.

In above-mentioned formula (A), l and h are preferably 1 or 2 independently.

According to a first alternative, the hydrophilic group of the cationic surfactant is a linear-chain or branched alkyl group having 1 to 12 carbon atoms and interrupted and/or substituted by at least one cationic group. As examples of such cationic surfactants the following can be cited:

1) (LiPO)—(CH₂)ₘ₁—NR₃₀R₃₁R₃₂, where Lipo is a lipophilic group such as defined above, m1 is 1 or 2 and R₃₀, R₃₁ and R₃₂ are independently H, Me or —CH₂—CH₂—OH,

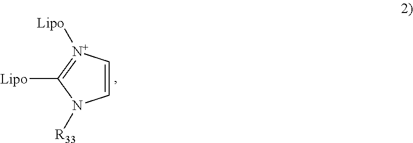

where each Lipo is independently a lipophilic group such as defined above and R33 is H, Me or —CH₂—CH₂—OH,

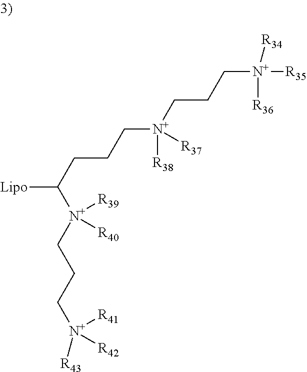

where Lipo is a lipophilic group such as defined above, and R₃₄, R₃₅, R₃₆, R₃₇, R₃₈, R₃₉, R₄₀, R₄₁, R₄₂ and R₄₃ are independently H, Me or —CH₂—CH₂—OH.

In one embodiment, the cationic surfactant is selected from among:
N[1-(2,3-dioleyloxyl)propyl]-N,N,N-trimethylammonium (DOTMA),
1,2-dioleyl-3-trimethylamonium-propane (DOTAP),
N-(2-hydroxyethyl)-N,N-dimethyl-2,3-bis(tetradecyloxy-1-propananium) (DMRIE),
1-[2-(oleoyloxy)ethyl]-2-oleyl-3-(2-hydroxyethyl)imidazolinium (DOTIM), and
dioctadecylamidoglycylspermine (DOGS) (in protonated form),
   and is preferably 1,2-dioleyl-3-trimethylamonium-propane (DOTAP).

According to a second alternative, the hydrophilic group of the cationic surfactant is a hydrophilic polymeric group comprising at least one cationic group.

When the hydrophilic group of the cationic surfactant is polymeric, the cationic group(s) may be terminal or pendant groups. For example:
when the hydrophilic polymeric group is a poly(ethylene oxide), the cationic group(s) are generally positioned on a terminal group at the end of the poly(ethylene oxide) chain;
when the hydrophilic polymeric group is dextran or cellulose, the cationic group(s) are generally positioned on a terminal group at the end of the polysaccharide chain;
when the hydrophilic polymeric group is chitosan, the cationic group(s) are generally a pendant group, in particular —NH₃⁺ groups present in an acid medium on chitosan.

In one embodiment, the cationic group(s) is/are a terminal group. The pendant groups of adjacent anionic surfactants on the surface of the droplets of the dispersed phase repel one another via electrostatic interactions and, as a result, the formulations comprising cationic surfactants in which the Hydro group comprises pendant groups are generally less stable.

In another embodiment, the cationic group(s) is/are pendant group(s). It is advantageously possible to use a cationic surfactant in which the hydrophilic group comprises several pendant cationic groups, and hence to obtain a formulation with greater positive charge and which will better allow the complexing of negative species such as the nucleotide sequences able to modulate endogenous mechanisms of interfering RNA.

The preferred hydrophilic polymeric group is a radical of a poly(ethylene oxide) typically comprising 3 to 500 ethylene oxide units, preferably 20 to 200 ethylene oxide units, and comprising at least one cationic group.

Therefore in one embodiment, the cationic surfactant has one of the following formulas:

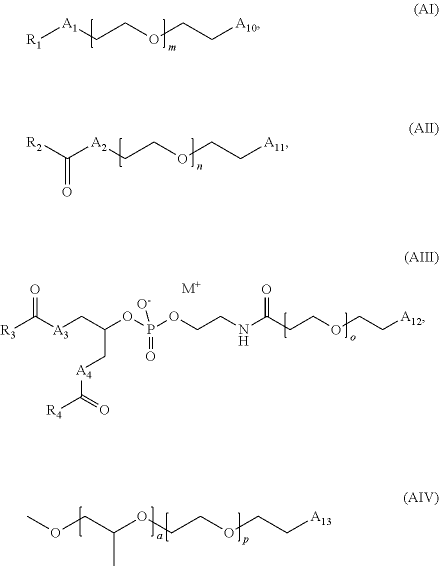

where:
R₁, R₂, R₃ and R₄ are independently a linear hydrocarbon chain having 11 to 23 carbon atoms;
A₁, A₂, A₃ and A₄ are O or NH;
m, n, o and p independently represent integers from 3 to 500, preferably 20 to 200; and
a is an integer from 20 to 120;
M is H or a cation;
A₁₀, A₁₁, A₁₂ and A₁₃ are independently a —⁺NR₂₀R₂₁R₂₂ group where R₂₀, R₂₁ and R₂₂ independently represent H, Me or —CH₂—CH₂—OH.

In one embodiment, in formula (AII), A₁₁ represents —⁺NH₃, and the cationic surfactant has the following formula:

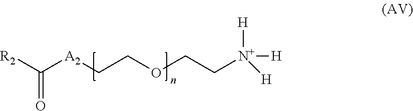

where A₂, R₂ and n are such as defined above. Preferably in formula (AII), R₂ represents C₁₇H₃₅.

Without wishing to be bound by any particular theory, it would seem that the presence of the hydrophilic polymeric group allows:
stabilisation of the formulation; and
protection of the nucleotide sequences able to modulate endogenous mechanisms of interfering RNA located on the surface of the droplets against the proteins of the medium in which the formulation is administered/used, and hence protection against degradation by these proteins of the nucleotide sequences able to modulate endogenous mechanisms of interfering RNA.

According to a third alternative, the formulation of the invention comprises at least two cationic surfactants, of which:
one is selected from among:
N[1-(2,3-dioleyloxyl)propyl]-N,N,N-trimethylammonium (DOTMA);
1,2-dioleyl-3-trimethylamonium-propane (DOTAP);
N-(2-hydroxyethyl)-N,N-dimethyl-2,3-bis(tetradecyloxy-1-propananium) (DMRIE);
1-[2-(oleoyloxy)ethyl]-2-oleyl-3-(2-hydroxyethyl)imidazolinium chloride (DOTIM); and
dioctadecylamidoglycylspermine (DOGS),

and is preferably 1,2-dioleyl-3-trimethylamonium-propane (DOTAP), and
the other is a cationic surfactant comprising:
   at least one lipophilic group selected from among:
      an R or R—(C═O)— group, where R is a linear hydrocarbon chain having 11 to 23 carbon atoms;
      an ester or an amide of fatty acids having 12 to 24 carbon atoms, and phosphatidylethanolamine, such as distearyl phosphatidylethanolamine (DSPE); and
      a poly(propylene oxide), and
   a hydrophilic polymeric group comprising at least one cationic group, the said polymeric group being selected from among:
      a poly(ethylene oxide) typically comprising 3 to 500 units of ethylene oxide, preferably 20 to 200 units of ethylene oxide, and comprising at least one cationic group;
      a polysaccharide, such as dextran, cellulose or chitosan;
      a polyamine, such as a chitosan or polylysine,

and is preferably a poly(ethylene oxide) comprising at least at least one cationic group.

In one embodiment the formulation of the invention, as cationic surfactants, comprises:
1,2-dioleyl-3-trimethylamonium-propane; and
a cationic surfactant comprising:
   at least one lipophilic group selected from among:
      an R or R—(C═O)— group, where R is a linear hydrocarbon chain having 11 to 23 carbon atoms;
      an ester or amide of fatty acids having 12 to 24 carbon atoms, and phosphatidylethanolamine such as distearyl phosphatidylethanolamine (DSPE); and
   a poly(ethylene oxide) typically comprising 3 to 500 units of ethylene oxide, preferably 20 to 200 units of ethylene oxide, and comprising at least one cationic group.

In one embodiment the formulation of the invention, as cationic surfactants, comprises:
1,2-dioleyl-3-trimethylamonium-propane; and
a compound of formula (AII) such as defined above, in particular of formula (AV).

The cationic surfactant is positioned in the crown part of the droplets of the formulation. It is linked via electrostatic interactions to the nucleotide sequences able to modulate endogenous mechanisms of RNA interference and allows the siRNAs to be maintained on the surface of the droplets.

The formulation comprises 15 to 70 mole % of at least one cationic surfactant relative to the whole (amphiphilic lipid/cationic surfactant/co-surfactant/optional helper lipid). Below 15%, the formulation does not contain sufficient positive charges and subsequent complexing of the <<premix>> formulation with the nucleotide sequences (negatively charged) is insufficient. Over and above 70%, the formulations are not stable and generally are even unable to be formulated (the forming of the nanoemulsion is not possible since the droplets coalesce to form two phases), and the droplets generally become toxic for the cells.

These proportions are particularly adapted to obtain efficient complexing of the nucleotide sequences able to modulate endogenous mechanisms of interfering RNA, and hence good delivery and/or transfection.

Helper Lipid

In one embodiment, the formulation of the invention comprises a helper lipid which is able to facilitate cytosolic release by destabilising the endosomal membrane. Preferably this lipid is dioleylphosphatidylethanolamine (DOPE).

This lipid allows the promoting of endosomal escape of the droplets of the formulation of the invention, and hence of the nucleotide sequences able to modulate endogenous mechanisms of RNA interference contained therein, and generally improves the silencing efficacy of the gene of interest.

The lipid able to facilitate cytosolic release by destabilising the endosomal membrane is positioned in the crown part of the droplets of the formulation.

Amphiphilic Lipid

The formulation comprises at least one amphiphilic lipid positioned in the crown part of the droplets of the formulation.

To form a stable nanoemulsion, it is necessary to include in the composition at least one amphiphilic lipid as surfactant. The amphiphilic nature of the surfactant ensures the stabilising of the oil droplets in the continuous aqueous phase. Below 5 mole % of amphiphilic lipid relative to the whole (amphiphilic lipid/cationic surfactant/co-surfactant/optional helper lipid), the formulations are not stable, and generally are not even able to be formulated (the forming of the nanoemulsion is not possible since the droplets coalesce to form two phases).

In general, the formulation comprises 5 to 85 mole %, preferably 5 to 75 mole %, in particular 5 to 50 mole % and further particularly 8 to 30 mole % of amphiphilic lipid relative to the whole (amphiphilic lipid/cationic surfactant/co-surfactant/optional helper lipid).

The quantity of amphiphilic lipid advantageously contributes towards controlling the size of the dispersed phase of the nanoemulsion.

The amphiphilic lipids comprise a hydrophilic part and a lipophilic part. They are generally selected from among compounds whose lipophilic part comprises a saturated or unsaturated, linear or branched chain having 8 to 30 carbon atoms. They can be selected from among phospholipids, cholesterols, lysolipids, sphingomyelins, tocopherols, glucolipids, stearylamines, cardiolipins of natural or synthetic origin; molecules composed of a fatty acid coupled to a hydrophilic group va an ether or ester function such as the esters of sorbitan e.g. sorbitan monooleate and monolaurate marketed under the trade name Span® by Sigma; polymerised lipids; lipids conjugated to short chains of polyethylene oxide (PEG) such as the non-ionic surfactants sold under the trade names Tween® by ICI Americas, Inc. and Triton® by Union Carbide Corp.; sugar esters such as mono- and di-laurate, mono- and di-palmitate, sucrose mono- and distearate; the said surfactants can be used alone or in a mixture.

Phospholipids are the preferred amphiphilic lipids.

Lecithin is a particularly preferred amphiphilic lipid.

Solubilising Lipid

The formulation also comprises a solubilising lipid comprising at least one fatty acid glyceride contained in the dispersed phase of the nanoemulsion, more specifically in the core part of the droplets. This compound has the chief role of solubilising the amphiphilic lipid which is scarcely soluble in the dispersed phase of the nanoemulsion.

The solubilising lipid is a lipid having sufficient affinity for the amphiphilic lipid to allow solubilising thereof. Preferably the solubilising lipid is solid at ambient temperature.

If the amphiphilic lipid is a phospholipid, it may in particular be:
esters of fatty acid and fatty alcohol such as cetyl palmitate; or
derivatives of glycerol and in particular of glycerides obtained by esterification of glycerol with fatty acids.

The solubilising lipid used is advantageously selected in relation to the amphiphilic lipid used. It generally has a close chemical structure to ensure the sought-after solubilisation. It may be an oil or wax. Preferably the solubilising lipid is solid at ambient temperature (20° C.), but liquid at body temperature (37° C.).

The preferred solubilising lipids, in particular for the phospholipids, are the esters of fatty acids and fatty alcohol such as cetyl palmitate, or the glycerides of fatty acids in particular of saturated fatty acids, and particularly saturated fatty acids having 8 to 18 carbon atoms, more preferably 12 to 18 carbon atoms. Advantageously it is a mixture of different glycerides.

Preferably, they are glycerides of saturated fatty acids comprising at least 10% by weight of C12 fatty acids, at least 5% by weight of C14 fatty acids, at least 5% by weight of C16 fatty acids and at least 5% by weight of C18 fatty acids.

Preferably, they are glycerides of fatty acids comprising 0% to 20% by weight of C8 fatty acids, 0% to 20% by weight of C10 fatty acids, 10% to 70% by weight of C12 fatty acids, 5% to 30% by weight of C14 fatty acids, 5% to 30% by weight of C16 fatty acids and 5% to 30% by weight of C18 fatty acids.

Particularly preferred are mixtures of semi-synthetic glycerides solid at ambient temperature sold under the trade name Suppocire®NC by Gattefossé and approved for injection in man. N-type Suppocire® are obtained by direct esterification of fatty acids and glycerol. They are semi-synthetic glycerides of saturated C8 to C18 fatty acids of which the quali-quantitative composition is given in the Table below.

The aforementioned solubilising lipids allow a formulation to be obtained in the form of an advantageously stable nanoemulsion. Without wishing to be bound by any particular theory, it is assumed that the aforementioned solubilising lipids allow the obtaining of droplets in the nanoemulsion which have an amorphous core. The core thus obtained has higher internal viscosity without exhibiting any crystallinity. Crystallisation is harmful for the stability of the nanoemulsion since it generally leads to aggregation of the droplets and/or to expelling of the encapsulated molecules outside the droplets. These physical properties promote the physical stability of the nanoemulsion.

The amount of solubilising lipid may vary extensively in relation to the type and amount of amphiphilic lipid contained in the dispersed phase. In general, the core of the droplets (comprising the solubilising lipid, optional oil, optional imaging agent, optional therapeutic agent if it is lipophilic) comprises 1 to 100% by weight, preferably 5 to 80% by weight and more preferably 40 to 75% by weight of solubilising lipid.

Fatty acid composition of Suppocire® NC by Gattefossé

| Chain length | [weight %] |
|---|---|
| C8 | 0.1 to 0.9 |
| C10 | 0.1 to 0.9 |
| C12 | 25 to 50 |
| C14 | 10 to 24.9 |
| C16 | 10 to 24.9 |
| C18 | 10 to 24.9 |

Oil

The dispersed phase may also comprise one or more other oils contained in the core of the droplets.

The oils used preferably have a hydrophilic-lipophilic balance (HLB) of less than 8 and more preferably of between 3 and 6. Advantageously the oils are used without chemical or physical modification prior to the forming of the emulsion.

The oils are generally selected from among biocompatible oils, and in particular from among oils of natural origin (vegetable or animal) or synthetic. Among these oils particular mention can be made of oils of natural vegetable origin amongst which are included soybean, flax, palm, groundnut, olive, grape seed and sunflower seed oil; synthetic oils which particularly include triglycerides, diglycerides and monoglycerides. These oils may be first press, refined or inter-esterified oils.

The preferred oils are soybean oil and flax oil.

In general and when present the oil is contained in the core of the droplets (comprising the solubilising lipid, optional oil, optional imaging agent, optional therapeutic agent if it is lipophilic) in a proportion ranging from 1 to 80% by weight, preferably between 5 and 50% by weight and more preferably 10 to 30% by weight.

The dispersed phase may further contain other additives such as dyes, stabilisers, preserving agents or other active ingredients in suitable amounts.

Co-Surfactant

The formulation comprises a co-surfactant which allows stabilising of the nanoemulsion.

The co-surfactants which can be used in the formulations of the invention are generally water-soluble surfactants. They comprise at least one poly(ethylene oxide) chain comprising at least 25, in particular at least 30, preferably at least 35 units of ethylene oxide. The number of ethylene oxide units is generally lower than 500.

Formulations comprising a co-surfactant comprising a poly(ethylene oxide) chain comprising fewer than 25 units of ethylene oxide are not stable. In general, it is not even possible to prepare the nanoemulsion.

These numbers of units are particularly adapted to prevent leakage of the nucleotide sequences, able to modulate endogenous mechanisms of interfering RNA, outside the droplets.

The inventors have effectively observed that a formulation not comprising a co-surfactant is not sufficiently stable.

In addition, without wishing to be bound by any particular theory, it would seem that the presence of the chain composed of ethylene oxide units in the co-surfactant provides protection of the nucleotide sequences able to modulate endogenous mechanisms of interfering RNAs located on the surface of the droplets against the proteins of the medium in which the formulation is administered/used, and hence against degradation of the said nucleotide sequences by these proteins.

As examples of co-surfactants particular mention can be made of the conjugated compounds polyethylene glycol/phosphatidyl-ethanolamine (PEG-PE), the ethers of fatty acid and of polyethylene glycol such as the products sold under the trade names Brij® (for example Brij® 35, 58, 78 or 98) by ICI Americas Inc., the esters of fatty acid and of esters polyethylene glycol such as the products sold under the trade names Myrj® by ICI Americas Inc. (for example Myrj® 45, 52, 53 ou 59) and the block copolymers of ethylene oxide and propylene oxide such as the products sold under the trade names Pluronic® by BASF AG (for example Pluronic® F68, F127, L64, L61, 10R4, 17R2, 17R4, 25R2 or 25R4) or the products sold under the trade name Synperonic® by Unichema Chemie BV (for example Synperonic® PE/F68, PE/L61 or PE/L64).

Therefore the co-surfactant is contained both in the continuous aqueous phase and in the dispersed phase. The hydrophobic part of the co-surfactant inserts itself in the droplets of the dispersed phase, whilst the polyalkoxylated chains are in the continuous aqueous phase. In the present application, the described weight percentages of the dispersed phase are calculated when considering that the co-surfactant belongs to the dispersed phase.

The formulation comprises 10% to 55 mole % of co-surfactant relative to the whole (amphiphilic lipid/cationic surfactant/co-surfactant/optional helper lipid). Below 10% the formulations are not stable and in general cannot even be formulated (the formation of the nanoemulsion is not possible since the droplets coalesce to form two phases). Over and above 55%, the subsequent complexing of the <<premix>> formulation with the nucleotide sequences does not take place probably because the positive charges of the cationic surfactant are masked by the poly(ethylene oxide) chains of the co-surfactant, and hence are more accessible for binding via electrostatic bonding with the nucleotide sequences.

The co-surfactant may also exhibit other effects in the envisaged application of the nanoemulsion.

According to one embodiment, the dispersed phase of the nanoemulsion is grafted on the surface with molecules of interest such as biological ligands, to increase the specific targeting of an organ. Said grafting allows specific recognition of some cells or some organs by promoting the internalisation of the droplets of the formulation of the invention by the target cells which express the surface receptor. It is therefore possible to transfect cells known to be resistant to prior art transfecting agents. Preferably, surface grafting is performed by coupling the molecules of interest or their precursors with an amphiphilic compound in particular with the co-surfactant. The nanoemulsion then comprises a grafted co-surfactant. In this case, the co-surfactant acts as spacer allowing the accommodating of the molecules of interest on the surface.

For example, the molecules of interest may be:
targeting biological ligands such as antibodies, peptides, saccharides, aptamers, oligonucleotides or compounds such as folic acid;
a stealth agent: an added entity to impart stealth to the nanoemulsion against the immune system, to increase its circulation time in the body and to slow down the elimination thereof.

For example when the biological ligand is a peptide comprising one or more cysteins, grafting to the alkylene oxide chain of the surfactant can be ensured by thiol maleimide coupling.

Imaging Agent

In one embodiment, the formulation comprises an imaging agent which advantageously allows visualisation of the distribution of the droplets in the cells or patient's body, and hence the distribution of the nucleotide sequences able to modulate endogenous mechanisms of RNA interference.

The imaging agent may be used in particular in imaging of the following type:
Positron Emission Tomography (PET) (the imaging agent possibly being a compound comprising a radionuclide such as ¹⁸F, ¹¹C, a chelate of metal cations ⁶⁸Ga, ⁶⁴Cu),
Single Photon Emission Computed Tomography (SPECT) (the imaging agent possibly being a compound comprising a radionuclide for example ¹²³I, or a chelate of ^{99m}Tc or ¹¹¹In),
Magnetic Resonance Imaging (MRI) (the imaging agent possibly being a chelate of gadolinium or a magnetic nanocrystal such as a nanocrystal of iron oxide, manganese oxide of iron-platinum FePt),
Optical imaging or X-ray imaging (the imaging agent possibly being a lipophilic fluorophore or a contrast agent, for example an iodine molecule such as iopamidol, amidotrizoate, or gold nanoparticles).

Preferably the imaging agent is a lipophilic fluorophore allowing the performing of optical imaging.

The type of lipophilic fluorophore(s) used is not critical provided that they are compatible with in vivo imaging (i.e. they are biocompatible and non-toxic). Preferably the fluorophores used as imaging agent absorb and emit in the visible or near infrared. For non-invasive imaging, in a tissue or living body (animal, man) the preferred fluorophores absorb and emit in the near infrared. For best passing through the tissues of the excitation light and the light emitted by the fluorophore, the most suitable fluorophores are those absorbing and emitting in the near infrared i.e. a wavelength of between 640 and 900 nm.

As lipophilic fluorophore it is possible to cite the compounds described in Chapter 13 (“Probes for Lipids and Membranes”) in the InVitrogen catalogue. More specifically, as fluorophore mention can particularly be made of indocyanine green (ICG), the analogues of fatty acids and phospholipids functionalised by a fluorescent group such as the fluorescent products sold under the trade names Bodipy (R) for example Bodipy (R) 665/676 (Ex/Em.); the lipophilic derivatives of carbocyanines such as 1,1′-dioctadecyl-3,3,3′,3′-tetramethylindodicarbocyanine perchlorate (DiD) sold for example under the reference D-307, 3,3′-dihexadecyloxacarbocyanine perchlorate (DiO) sold for example under the reference D1125, 1,1′-dihexadecyl-3,3,3′,3′-tetramethylindocarbocyanine perchlorate (Dil) sold for example under the reference D384; fluorescent probes derived from sphingolipids, from steroids or from lipopolysaccharides such as the products sold under the trade names BODIPY® TR ceramids, BODIPY® FL C5-lactosylceramide, BODIPY® FL C5-ganglioside, BODIPY® FL cerebrosides; the amphiphilic derivatives of cyanines, of rhodamines, of fluoresceins or coumarins such as octadecyl rhodamine B, octadecyl ester of fluorescein and 4-heptadecyl-7-hydroxycoumarin; and diphenylhexatriène (DPH) and derivatives thereof; all these products being sold by Invitrogen.

According to one preferred embodiment of the invention, the fluorophore is indocyanine green, 1,1′-dioctadecyl-3,3,3′,3′-tetramethylindodicarbocyanine perchlorate, 3,3′-dihexadecyloxacarbocyanine perchlorate, or 1,1′-dihexadecyl-3,3,3′,3′-tetramethylindocarbocyanine perchlorate.

Therapeutic Agent

The formulation of the invention may comprise a therapeutic agent.

The therapeutic agents able to be encapsulated in the nanoemulsion of the invention particularly contain the active ingredients acting via chemical, biological or physical route. These may therefore be pharmaceutical active ingredients or biological agents such as DNA, proteins, peptides or antibodies or agents useful for physical therapy such as compounds useful for heat therapy, compounds releasing singlet oxygen when excited by light and used for phototherapy, and radioactive agents. Preferably they are active ingredients administered via parenteral route.

Depending on its lipophilic or amphiphilic affinity the therapeutic agent can be encapsulated by the dispersed phase or is positioned at the interface of the two phases.

The type of therapeutic agent encapsulated in the nanoemulsion is not particularly limited. However the nanoemulsion is of particular interest for scarcely soluble compounds which are difficult to formulate in conventional administration systems, and for active ingredients used in phototherapy for which the quantum yield can be maintained.

On account of the mild conditions of the preparation method, the described formulation is of particular interest for the encapsulating of therapeutic agents which are degraded at high temperature.

Among the pharmaceutical active ingredients of interest as therapeutic agents, particular mention can be made of the agents used in the treatment of AIDS, the agents used in the treatment of heart disease, analgesics anaesthetics, anorexigenics, anthelmintics, antiallergics, antianginals, antiarrhythmics, anticholinergics, anticoagulants, antidepressants, antidiabetics, antidiuretics, antiemetics, anticonvulsants, antifungals, antihistaminics, antihypertensives, anti-inflammatories, anti-migraines, antimuscarinics, antimycobacterials, anticancer drugs including antiparkinsons, antithyroid drugs, antivirals, astringents, blocking agents, blood products, blood substitutes, cardiac inotropic agents, cardiovascular agents, central nervous system agents, chelators, chemotherapy agents, hematopoietic growth factors, corticosteroids, antitussives, dermatological agents, diuretics, dopaminergic drugs, elastase inhibitors, endocrine agents, ergot alkaloids, expectorants, gastro-intestinal agents, genitourinary agents, growth hormone triggering factor, growth hormones, hematologic agents, hematopoietic agents, hemostatics, hormones, immunosuppressives, interleukins, analogues of interleukins, lipid regulating agents, gonadoliberin, muscle relaxants, narcotic antagonists, nutrients, nutritive agents, oncology therapy, organic nitrates, vagomimetics, prostaglandins, antibiotics, renal agents, respiratory agents, sedatives, sexual hormones, stimulants, sympathomimetics, systemic anti-infectious agents, tacrolimus, thrombolytic agents, thyroid agents, treatments for attention disorders, vasodilators, xanthines, cholesterol-reducing agents. Particularly targeted are anticancer agents such as taxol (paclitaxel), doxorubicin and cisplatin.

Among the physical or chemical agents, particularly cited are radioactive isotopes and photo-sensitizers.

Among the photo-sensitizers, mention can be made of those belonging to the class of tetrapyrroles such as the porphyrins, bacteriochlorins, phthalocyanines, chlorines, purpurins, porphycenes, pheophorbides, or those belonging to the class of texaphyrins or hypericins. Among the first-generation photo-sensitizers mention can be made of hemato-porphyrin and a mixture of derivatives of hemato-porphyrin (HpD) (sold under the trade name Photofrin® by Axcan Pharma). Among the second-generation photo-sensitizers mention can be made of meta-tetra-hydroxyphenyl chlorin (mTHPC; trade name Foscan®, Biolitec AG) and the cycle A monoacid derivative of benzoporphyrin (BPD-MA sold under the trade name Visudyne® by QLT and Novartis Opthalmics). The formulations of second generation photo-sensitizers which associate a molecule therewith (lipid, peptide, sugar etc.) termed a transporter allowing their selective conveying to the tumour tissue are called third generation photo-sensitizers.

Among the biological agents, particular mention is made of oligonucleotides, DNA, RNA, siRNA, peptides, proteins and saccharides.

Evidently the therapeutic agent can be formulated directly in its active form or in prodrug from. Also it is envisaged that several therapeutic agents are able to be formulated in association within the nanoemulsion.

The amount of therapeutic agent is dependent on the targeted application and the type of agent. However it is generally sought to formulate the nanoemulsion with a maximum concentration of therapeutic agent, in particular if they are scarcely soluble therapeutic agents to limit the volume and/or period of administration to the patient.

Yet it has been found that the presence of the solubilising lipid in the dispersed phase allows the incorporation of a high quantity of compounds even hydrophobic or amphiphilic compounds.

Core Proportion in the Droplets

In general the mole proportion of components in the core of the droplets relative to the components of the droplets is 10 to 80%, in particular 25 to 75%, preferably 33.35 to 73.99%. In other words, the mole proportion (mol/mol) of the whole (solubilising lipid/optional oil/optional imaging agent/optional lipophilic therapeutic agent) relative to the dispersed phase (i.e. to all the components of the dispersed phase) is generally 10 to 80%, in particular 25 to 75%, preferably 33.35 to 73.99%.

In general the weight proportion (wt/wt) of the core components of the droplets relative to the components of the droplets is 10 to 60%, in particular 20 to 60%, preferably 23.53 to 59.51%. In other words, the weight proportion of the whole (solubilising lipid/optional oil/optional imaging agent/optional lipophilic therapeutic agent) relative to the dispersed phase (i.e. to all the components of the dispersed phase) is generally 10 to 60%, in particular 20 to 60%, preferably 23.53 to 59.51%.

These mole and/or weight proportions are particularly adapted so that the <<premix>> formation is stable on storage, in particular stable when stored more than 28 days at 40° C., even more than 300 days at 40° C. Stability can measured in particular by monitoring the size of the droplets, their polydispersity index and/or their zeta potential (e.g. by dynamic light scattering using apparatus of type ZetaSizer, Malvern). This stability of the <<premix>> formulation is important for the envisaged applications. As detailed below, the <<premix>> formulation is used to prepare the <<final>> formulation comprising a nucleotide sequence able to modulate endogenous mechanisms of RNA interference. It is of particular advantage to be able to store the <<premix>> formulation and to perform complexing with the said sequence just before use of the <<final>> formulation.

Aqueous Phase

The aqueous phase of the nanoemulsion used in the invention is preferably formed of water and/or a buffer such as a phosphate buffer e.g. PBS (“Phosphate Buffer Saline”) or a saline solution in particular sodium chloride.

According to one embodiment, the continuous aqueous phase also comprises a thickening agent such as glycerol, a saccharide, oligosaccharide or polysaccharide, a gum or a protein, preferably glycerol. The use of a continuous phase of higher viscosity facilitates emulsification and thereby allows a reduction in sonication time.

The aqueous phase advantageously comprises 0 to 50% by weight, preferably 1 to 30% by weight and more particularly 5 to 20% by weight of thickening agent.

Evidently the aqueous phase may further contain other additives such as dyes, stabilisers and preserving agents in suitable amounts.

The proportion of dispersed phase and aqueous phase is most variable. However, most often the nanoemulsions are prepared with 1 to 50%, preferably 5 to 40% and more particularly 10 to 30% by weight of dispersed phase and 50 to 99%, preferably 60 to 95% and more particularly 70 à 90% by weight of aqueous phase.

Nanoemulsion in Gel Form

In one embodiment, the viscosity of the formulation is higher than 1 poise (0.1 Pa·s) at 25° C.

In this embodiment, the nanoemulsion used is in the form of a <<gel>>. By the term <<gel>> is usually meant a two-phase solid-liquid system that is thermodynamically stable, formed of a three-dimensional continuous interpenetrating double network, one solid and the second liquid. A said gel is a two-phase liquid-solid system in which the solid network retains the liquid phase. Although gels may be considered as solids, they exhibit properties particular to solids (structural rigidity, elasticity on deformation) as well as to liquids (vapour pressure, compressibility pressure and electric conductivity).

For a nanoemulsion in gel form, the three-dimensional network is formed by the droplets, the interstices between the droplets being filled with continuous phase. The bonds between the units of the network, namely the droplets, are chiefly based on electrostatic interactions (ion pairs). These electrostatic interactions mainly exist between the nucleotide sequences able to modulate endogenous mechanisms of RNA interference and the cationic surfactants of adjacent droplets.

A nanoemulsion in gel form therefore displays resistance to pressure and is capable of maintaining a defined shape which can be of advantage in relation to the desired administration shape and/or route.

To evidence that the nanoemulsion is in gel form rheological studies can be conducted to evaluate viscoelastic properties and/or more structural studies demonstrating the bonds between the droplets forming the three-dimensional network (X-ray diffraction, neutrons . . . ). A nanoemulsion is gel form has higher viscosity and coefficient of elasticity than a liquid nanoemulsion. The nanoemulsion in gel form, in relation to droplet concentration and hence to the weight fraction in the dispersed phase can be in the viscous liquid state, viscoelastic solid state or elastic solid state. Compared with the aqueous dispersing phase wherein the viscosity is close to that of water (1 mPa·s at 25° C.), the nanoemulsion is considered to be a viscous liquid when its viscosity is 10 times higher than that of water i.e. >10 mPa·s at 25° C. Also, with rheological measurement of G′ (shear storage modulus) and G″ (shear loss modulus) it is considered that a nanoemulsion is in the form of a viscous liquid when G″>G′. When G′ draws close to G″, a nanoemulsion is in a viscoelastic solid state. When G″<G′, the state is an elastic solid state. In this embodiment, the nanoemulsion is preferably in the viscous liquid state or viscoelastic solid state since viscosity is sufficiently moderate in these states to allow applications involving administration via injection. Viscosity and the coefficient of elasticity can be measured using a cone/plate rheometer or Couette rheometer. The viscosity of a liquid nanoemulsion is generally lower than 1 poise, even often lower than 0.01 poise. The nanoemulsion used in this embodiment generally has viscosity higher than 1 poise, and may have viscosity as high as that of a solid (higher than 1000 poises). The nanoemulsion of the present invention generally has viscosity of 1 to 1000 poises, preferably 1 to 500 poises and further preferably between 1 and 200, these values being given at 25° C. Viscosity higher than 1 poise is adapted so that the droplets of the dispersed phase form a three-dimensional network inside the continuous phase. It has been ascertained that below 1 poise the droplets are generally insufficiently close to one another. Over and above 1000 poises, a near-solid system is obtained. The nanoemulsion is then too viscous and difficult to use. Similarly whereas the coefficient of elasticity is generally lower than 10 for a liquid nanoemulsion, the coefficient of elasticity of a nanoemulsion in gel form is generally higher than 10. Structural studies, in particular X-ray or neutron diffraction, also allows differentiation between the organisation of a liquid nanoemulsion and the organisation of a nanoemulsion in gel form. The peaks of the diffractogram obtained for a liquid nanoemulsion are characteristic of the structure of the droplets in the dispersed phase (large diffraction angles characteristic of short distances) whereas the peaks of the diffractogram for a nanoemulsion in gel form are characteristic not only through the structure of the droplets (large angles of diffraction characteristic of short distances) but also through the organisation of these droplets in a three-dimensional network (small angles of diffraction characteristic of longer distances).

The nanoemulsion used in this embodiment of the invention is advantageously in dispersible gel form i.e. the droplets forming the three-dimensional network can be released into the continuous phase under certain conditions via <<degelling>> of the gel system, also called <<breakdown>> in the present application. Breakdown is observed by adding a continuous phase to the gel, by contacting with physiological fluids when administering the nanoemulsion or by temperature increase The adding of continuous phase leads to a difference in osmotic pressure between the inside of the gel and the continuous phase. The system will therefore tend to decrease going as far as cancelling out this difference in osmotic pressure by releasing droplets in the excess continuous phase until a homogeneous concentration of droplets is obtained in the entire volume of the continuous phase. Similarly a sufficient temperature increase of the system amounts to imparting thermal energy to the different droplets that is higher than the energies contained in the bonds for example the hydrogen bonds, and therefore breaks these bonds and releases the droplets of the three-dimensional network. These temperatures are dependent on the composition of the gel and more particularly on the size of the droplets and the length of the polyalkoxylated chains of the co-surfactant. The breakdown of the nanoemulsion in gel form can be monitored by X-ray diffraction, by differential scanning calorimetry (DSC) or nuclear magnetic resonance (NMR). When monitoring breakdown of the nanoemulsion in gel form by X-ray diffraction, a change in the spectrogram is observed i.e. a decrease in the intensity of the small angles (characteristic of the organisation of the droplets in a three-dimensional network) (as described in Matija Tomsic, Florian Prossnigg, Otto Glatter ‘Journal of Colloid and Interface Science’ Volume 322, Issue 1, 1 Jun. 2008, Pages 41-50). The breakdown can also be monitored by DSC. A peak is seen in the thermogram during the transition of nanoemulsion in gel form/liquid nanoemulsion as the temperature rises. Finally a NMR study can also be used to monitor breakdown by measuring the diffusion coefficient associated with each droplet distinguishing a liquid nanoemulsion from a nanoemulsion in gel form. The diffusion coefficient is most significantly reduced for a nanoemulsion in gel form (it is then generally lower than 0.01 μm²/s), when the system is blocked (WESTRIN B. A.; AXELSSON A.; ZACCHI G. ‘Diffusion measurement in gels’, Journal of controlled release 1994, vol. 30, n^{o} 3, pp. 189-199).

Emulsion in which the Droplets are Covalently Bonded Together

In one embodiment, the formulation comprises a surfactant of following formula (I):

(L₁-X₁—H₁—Y₁)ᵥ-G-(Y₂—H₂—X₂-L₂)_{w} (I),

where:
L₁ and L₂ are independently lipophilic groups;
X₁, X₂, Y₁, Y₂ and G are independently a linkage group;
H₁ and t H₂ rare independently hydrophilic groups comprising a polyalkoxylated chain;
v and w are independently an integer from 1 to 8,
   and the droplets in the dispersed phase are covalently bonded together by the surfactant of formula (I).

The surfactant of formula (I) is partly contained in the continuous aqueous phase and partly in the dispersed phase. The surfactant of formula (I) in fact comprises two lipophilic groups (L₁ and L₂) and two hydrophilic groups (H₁ and H₂). The hydrophilic groups are mostly located on the surface of the droplets, in the continuous aqueous phase, whereas the lipophilic groups are located inside the droplets of the formulation.

More specifically the lipophilic group L₁ is located inside some droplets and the group L₂ in adjacent droplets. The droplets are therefore covalently linked together by the group —(X₁—H₁—Y₁)ᵥ-G-(Y₂—H₂—X₂)_{w}— of the surfactant of formula (I).

The groups X₁ and X₂ are linkage groups linking the lipophilic and hydrophilic groups. Group G is a linkage group between the two parts [lipophilic-hydrophilic] of the surfactant of formula (I). The groups Y₁ and Y₂ are linkage groups linking group G to these two parts [lipophilic-hydrophilic].

The formulation of this embodiment can advantageously be shaped (e.g. by placing in a mould or receptacle of given shape) and can remain in the desired shape depending on the desired application. This embodiment of the invention is therefore particularly adapted when the formulation is used in the form of a capsule, gel, ovule or patch.

In addition it resists dilution to an aqueous phase. More specifically, when an aqueous phase is added to this formulation, the formulation maintains its shape and is not diluted. In the medium, first the formulation comprising the droplets can be seen and secondly an aqueous phase essentially free of droplets.

Without wishing to be bound by any particular theory, it would seem that these properties of this formulation can be accounted for through the presence of the covalent bonds between the droplets, which impart very strong cohesion thereto.

In one embodiment, in above-mentioned formula (I):
L₁ and L₂ are independently selected from among:
   an R or R—(C═O)— group, where R is a linear hydrocarbon chain having 11 to 23 carbon atoms;
   an ester or amide of fatty acids having 12 to 24 carbon atoms and phosphatidylethanolamine such as distearyl phosphatidylethanolamine (DSPE); and
   a poly(propylene oxide); and/or
X₁, X₂, Y₁ and Y₂ are independently selected from among:
   a single bond;
   a Z group selected from among —O—, —NH—, —O(OC)—, —(CO)O—, —(CO)NH—, —NH(CO)—, —O—(CO)—O—, —NH—(CO)—O—, —O—(CO)—NH— and —NH—(CO)—NH;
   an Alk group being an alkylene comprising 1 to 6 carbon atoms; and
   a Z-Alk, Alk-Z, Alk-Z-Alk or Z-Alk-Z group where Alk and Z are such as defined above and where the two Z groups of the Z-Alk-Z group are the same or different; and/or
H₁ and H₂ are independently selected from among a poly(ethylene oxide) typically comprising 3 to 500 units of ethylene oxide, preferably 20 to 200 units of ethylene oxide; and/or
G comprises at least one G′ group having one of the following formulas (the groups Y₁ and Y₂ being linked on the left and right of the formulas described below):

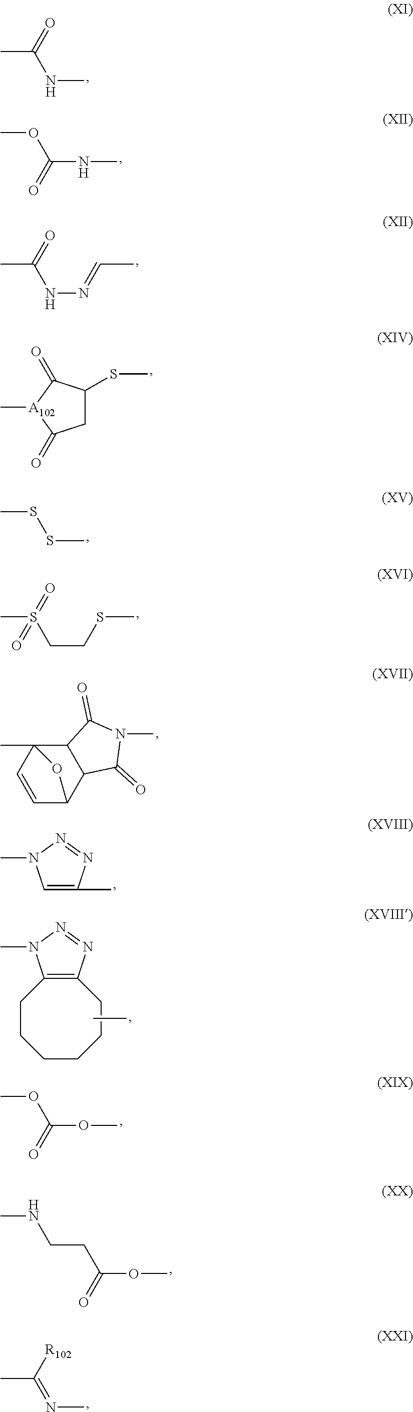

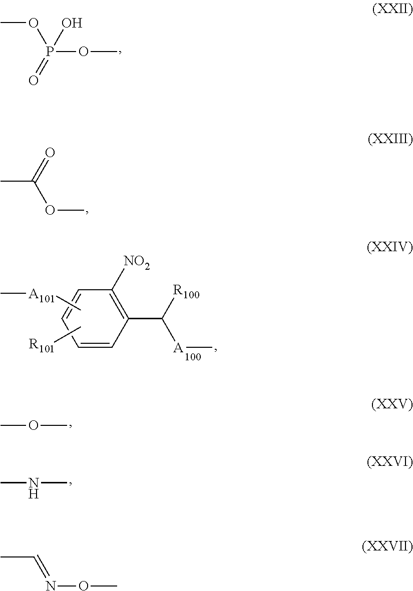

where A₁₀₂ represents CH or N, R₁₀₂ represents H or a linear hydrocarbon chain having 1 to 6 carbon atoms, A₁₀₁ represents —O—, —NH—(CO)— or —O—(CO)—, R₁₀₀ represents H or a methyl, A₁₀₀ represents —O— or —NH— and R₁₀₁ represents H, Me or —OMe.
   By the formula

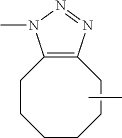

it is meant that the Y₂ group can be linked to any of the six atoms of the cyclooctyl and by the formula

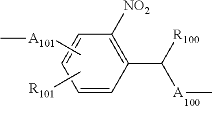

it is meant that the groups A₁₀₁ and R₁₀₁ can be linked to any of the four atoms of the phenyl.

In particular, v and w independently represent 1 or 2. Preferably v and w are 1.

The G group may comprise one or more G′ groups defined above.

Therefore in a first embodiment the G group is formed of one G′ group. In this embodiment, in formula (I), v and w represent 1.

In a second embodiment, the G group meets formula -G′-Y₃-G′- where:
Y₃ is a linkage group selected in particular from among:
   a single bond;
   a Z group selected from among —O—, —NH—, —O(OC)—, —(CO)O—, —(CO)NH—, —NH(CO)—, —O—(CO)—O—, —NH—(CO)—O—, —O—(CO)—NH— and —NH—(CO)—N;
   an Alk group being an alkylene having 1 to 6 carbon atoms; and
   a Z-Alk, Alk-Z, Alk-Z-Alk or Z-Alk-Z group where Alk and Z are such as defined above and where the two Z groups of the Z-Alk-Z group are the same or different;
each G′ group independently represents a group of above-mentioned formulas (XI) to (XXVI and preferably the two G′ groups of the formula -G′-Y₃-G′- are the same.
   In this embodiment in formula (I), v and w represent 1.
   This embodiment is of particular interest when the two G′ groups are the same and comprise a cleavable function. It is then sufficient to cleave only one of the two functions to break the covalent bonds between the droplets of the formulation.

In a third embodiment, the G group is a dendrimer comprising (v+w) G′ groups. The G group may in particular be a dendrimer comprising several G′ groups, such as a dendrimer comprising a polyamidoamine group (PAMAM). For example the G group may have one of the following formulas (XXX) to (XXXIII) which comprise:
4 G′ groups of formula (XXVI): v and w represent 2.
4 G′ groups of formula (XXIV) where R₁₀₁ represents —O-Me, A₁₀₁ represents —NH—, R₁₀₀ represents a methyl and A₁₀₀ represents —NH—. v and w represent 2.
4 G′ groups of formula (XIV). v and w represent 2.
16 G′ groups of formula (XXVI). v and w represent 8.

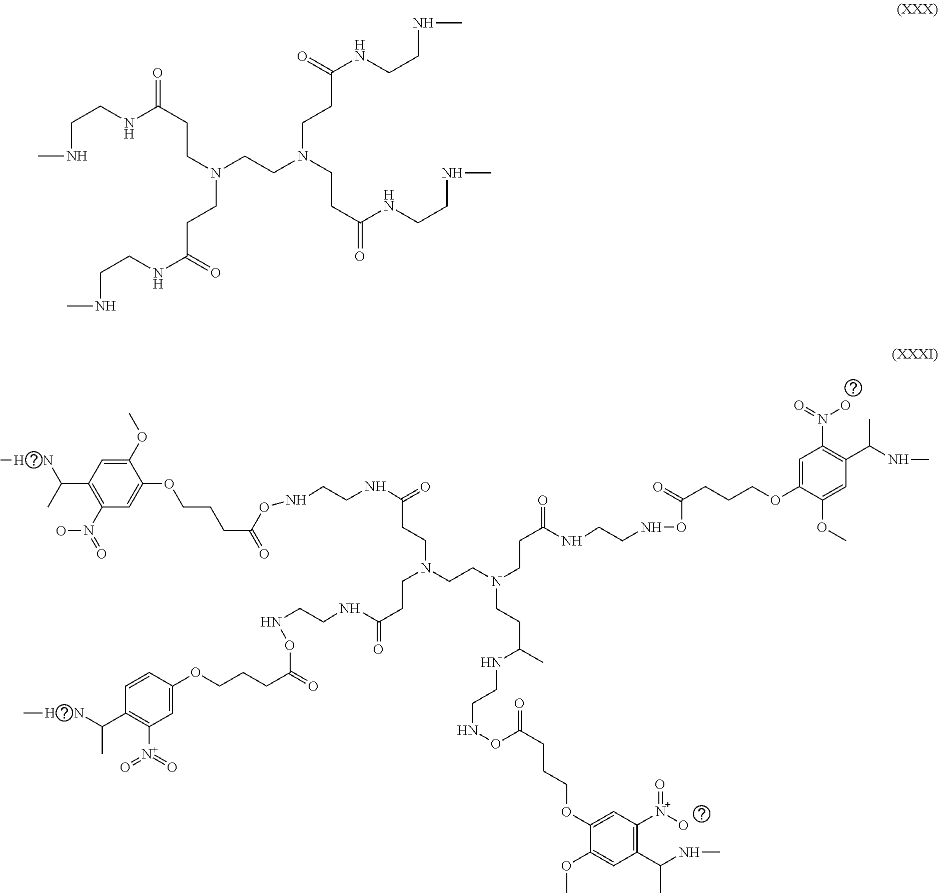

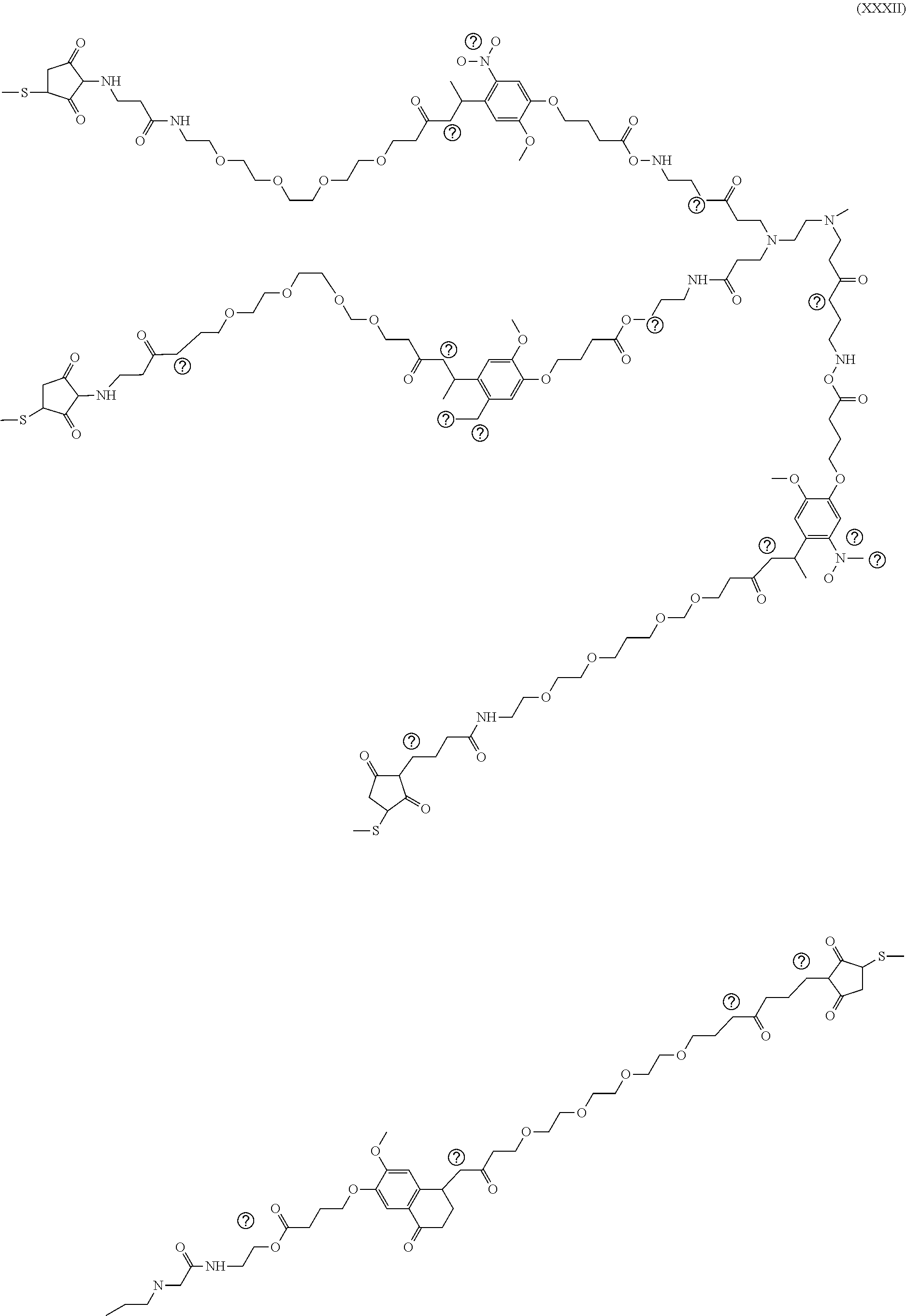

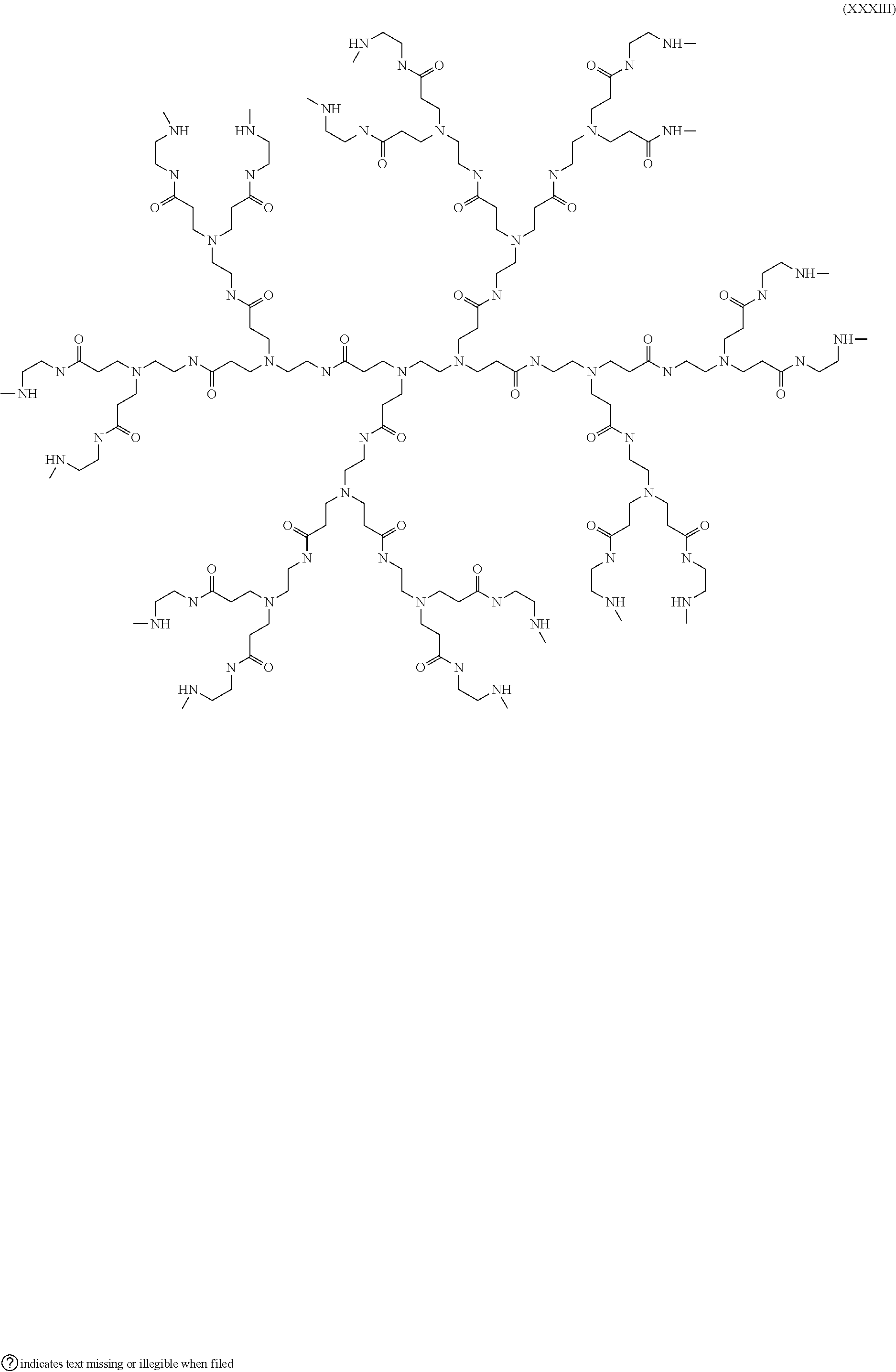

When L₁ and/or L₂ are an R—(C═O)— group where R represents a linear hydrocarbon chain having 11 to 23 carbon atoms, L₁ and/or L₂ represent groups derived from fatty acids having 12 to 24 carbon atoms.

By <<L₁ and L₂ represent an ester or amide of fatty acids having 12 to 24 carbon atoms and phosphatidylethanolamine>>, it is meant that they represent a group of formula

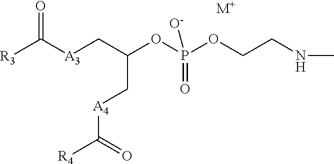

where:
R₃ and R₄ are independently a linear hydrocarbon chain having 11 to 23 carbon atoms;
A₃ and A₄ are 0 or NH; and
M is H or a cation.

Preferably, L₁ and L₂ are the same and/or X₁ and X₂ are the same and/or H₁ and H₂ are the same. Particularly preferred surfactants of formula (I) are those in which L₁ and L₂ are the same, X₁ and X₂ are the same, and H₁ and H₂ are the same. These surfactants are symmetrical compounds and are therefore generally easier to synthesize and hence less costly.

In one embodiment, in the abovementioned formula (I) the radicals L₁-X₁—H₁— and/or L₂-X₂—H₂— consist of one of the groups of the following formulas (the Y₁ or Y₂ group being linked on the right of the formulas described below):

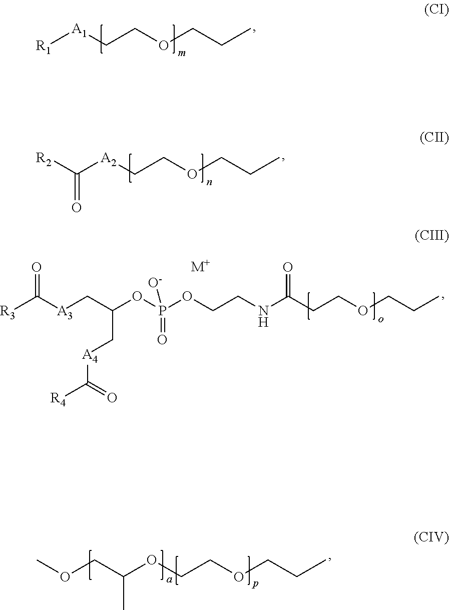

where:
R₁, R₂, R₃ and R₄ are independently a linear hydrocarbon chain having 11 to 23 carbon atoms;
A₁, A₂, A₃ et A₄ are O or NH;
m, n, o and p are independently integers from 3 to 500, preferably 20 to 200; and
a is an integer from 20 to 120;
M is H or a cation.

The radicals L₁-X₁—H₁— and/or L₂-X₂—H₂— of formula (CII) are preferred. They are easy to prepare (in particular by formation of an ester or amide between a fatty acid and a derivative of poly(ethylene glycol). Also a formulation comprising a surfactant comprising a radical L₁-X₁—H₁— and/or L₂-X₂—H₂— of formula (CII) can generally be prepared with a larger amount of this surfactant than a formulation comprising a surfactant comprising a L₁-X₁—H₁— and/or L₂-X₂—H₂-radical of formula (CIII). The greater the proportion of surfactant of formula (I) in the formulation the greater the cohesion between the droplets and the more the formulation maintains its shape and resists dilution. Therefore these two properties can be further enhanced with a formulation comprising a surfactant comprising a L₁-X₁—H₁— and/or L₂-X₂—H₂— radical of formula (CII).

The radicals L₁-X₁—H₁— and/or L₂-X₂—H₂— of formula (CII) where A₂ represents NH are particularly preferred since the surfactants comprising such radicals can prevent leakage outside the droplets of optionally present lipophilic agents of interest in more efficient manner than the surfactants comprising L₁-X₁—H₁— and/or L₂-X₂—H₂— radicals of formula (CII) where A₂ represents O.

In one embodiment, in formula (I), v and w represent 1, L₁ and L₂ are independently R—(C═O)—, where R is a linear hydrocarbon chain having 11 to 23 carbon atoms, H₁ and H₂ are independently poly(ethylene oxide) comprising 3 to 500 units of ethylene oxide, X₁ and X₂ represent —O— or —NH—, G is formed of a G′ group representing —S—S— (group of formula (XV) above) and Y₁ and Y₂ represent —CH₂—CH₂—NH—CO—CH₂—CH₂— (Alk-Z-Alk group above where Alk represents —CH₂—CH₂— and Z represents —NH—(CO)—) and the surfactant of the formulation then has following formula (I′):

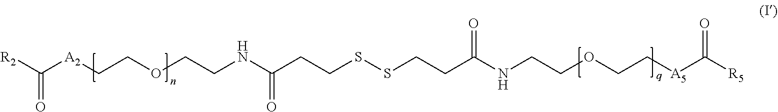

where:
R₂ and R₅ are independently a linear hydrocarbon chain having 11 to 23 carbon atoms preferably 17,
A₂ and A₅ represent 0 or NH, preferably NH, and
n and q are independently integers of 3 to 500, preferably 20 to 200.

In one embodiment, the groups H₁ and H₂ are independently selected from among a poly(ethylene oxide) comprising more than 3 units of poly(ethylene oxide) even more than 20 units, in particular more than 50 (in the above-mentioned formulas m, n, o, p and/or q are preferably higher than 3, even 20, in particular higher than 50).

In one embodiment the G group of the surfactant in formula (I) of the formulation comprises a function that is clevable, in particular at certain pH values (basic or acid pH), by enzymes, by light (visible light, ultraviolet or infrared) and/or over and above certain temperatures. In general the G group then includes a group G′ comprising a cleavable function.

For example:
the β-ketoaminoester function of the G group in formula (XX) is cleavable at acid pH (typically at around 5),
the disulfide function of the G group in formula (XV) is cleavable under ultraviolet or with enzymes such as thioreductases,
the amide function of the G group in formula (XI) is cleavable with enzymes such as proteases,
the phosphate function of the G group in formula (XXII) is cleavable with enzymes such as phosphatases,
the imine function of the G groups in formulas (XXI) and (XIII) are cleavable at acid pH or over and above certain temperatures,
the cyclohexene crown of the G group in formula (XVII) is cleavable over and above certain temperatures (via retro Diels-Alder),
the carbonate function of the G group in formula (XIX) and the carbamate function of group G in formula (XII) are cleavable at acid pH.

Persons skilled in the art, in the light of their general knowledge, know which functions are cleavable and under which conditions. It is notably within their reach to select the function of the G′ group of the surfactant in formula (I) so that it can be cleaved under the conditions encountered for administration of the formulation according to the invention.

Preferably the ratio of the weight of surfactant of formula (I) to the weight of the whole (surfactant of formula (I)/co-surfactant) is no lower than 15%. It has effectively been observed that such formulations are easier to prepare.

Size of the Droplets in the <<Premix>> Formulation

The droplets of the <<premix>> formulation defined above generally have a diameter of between 20 and 200 nm. In particular this diameter can be measured by dynamic light scattering on Malvern ZetaSizer apparatus.

It is possible to obtain droplets of more specific size by adapting the percentages of the components of the nanoemulsion.

For a formulation comprising droplets of size between 20 and 40 nm, preference is given to a formulation comprising at least 5 mole % of amphiphilic lipid, and:
25 to 45 mole % of co-surfactant (below 25 mole % the formulation may exhibit stability problems); and/or
15 to 50 mole % of cationic surfactant.

For a formulation comprising droplets of size between 40 and 100 nm, preference is given to a formulation comprising at least 5 mole % of amphiphilic lipid and:
45 to 50 mole % of co-surfactant (below 45 mole %, the formulation may exhibit stability problems. Above 50% the transfecting efficacy of the <<final>> formulation after complexing with the nucleotide sequences is lower); and/or
30 to 40 mole % of cationic surfactant (below 30 mole % the transfecting efficacy of the <<final>> formulation after complexing with the nucleotide sequences is lower. Over and above 40% the formulation may exhibit stability problems).

For a formulation comprising droplets of size between 130 and 175 nm, preference is given to a formulation comprising at least 5 mole % of amphiphilic lipid and 15 to 70 mole % of at least one cationic surfactant, and:
10 to 25 mole %, in particular 10 to 15% of co-surfactant.

The mole percentages of amphiphilic lipid, of cationic surfactant and of co-surfactant are relative to the whole (amphiphilic lipid/cationic surfactant/co-surfactant/optional helper lipid).

### [Formulation Comprising a Nucleotide Sequence Able to Modulate Endogenous Mechanisms of Interfering RNA] (Also Called <<Final Formulation>>)

Nucleotide Sequence Able to Modulate Endogenous Mechanisms of Interfering RNA

By complexing the <<premix>> formulation defined above with a nucleotide sequence able to modulate endogenous mechanisms of RNA interference, a <<final>> formulation is obtained useful for delivering the said sequences.

Therefore the formulation may additionally comprise a single or double strand nucleotide sequence, comprising fewer than 200 bases for a single strand nucleotide sequence, or fewer than 200 base pairs for a double strand nucleotide sequence, that is able to modulate endogenous mechanisms of interfering RNA (ribonucleic acid), such as:
a short or small interfering RNA-siRNA;
a locked nucleic acid-LNA;
a synthetic microRNA mimic called MicroRNA or miRNA.

In one embodiment the said nucleotide sequences is a siRNA.

A siRNA is a nucleotide sequence of double strand RNA. It is a natural or synthetic sequence.

A siRNA is able to target a transcript of interest i.e. the nucleotide sequence of one of the strands of the siRNA is complementary to the nucleotide sequence of the transcript of interest. The size of each strand of the double strand of the siRNA generally varies from 15 to 30 nucleotides, preferably 19 to 25 nucleotides, in particular 19 to 21 nucleotides. Two deoxythymidines are generally added to the 3′ portion of each of its strands to increase the stability thereof. Therefore a siRNA in which deoxythymidines have been grafted onto its 3′ portion does not depart from the definition of a siRNA according to the present application. A siRNA allows a reduction in the expression of a target protein by interfering with the messenger RNA encoding this protein.

In one embodiment the said nucleotide sequence is a locked nucleic acid.

A locked nucleic acid is a nucleotide sequence of single strand RNA and/or DNA of which at least one of the nucleic acids contains a methylene bridge between the hydroxyl at position 2 and the carbon atom 4 of ribose. It is a synthetic nucleotide sequence.

A locked nucleic acid is an inhibitor of microRNA and allows regulating of the expression of one or more target proteins the mRNA of which were in interference with the RNA sequences derived from the said microRNA. Regulation most often entails lifting of the inhibition of protein expression.

In one embodiment the said nucleotide sequence is a microRNA.

A microRNA is a nucleotide sequence of single strand RNA (in the order of one hundred bases). It is a synthetic nucleotide sequence.

A microRNA allows regulating of the expression of one or more target proteins by interference with one or more mRNAs respectively encoding these proteins. Regulation most often entails inhibition of the expression of the proteins.

The said nucleotide sequences are maintained on the surface of the droplets of the dispersed phase of the formulation by means of electrostatic interactions with the cationic surfactant. They are therefore located on the surface of the droplets at the crown part of the droplets on the hydrophilic side of the crown.

Apart from the optional binding of the siRNA to deoxythymidines mentioned above, the said nucleotide sequences are not chemically modified and they are not denatured. In particular, the said nucleotide sequences are not covalently bonded to the other components of the droplets. In particular the said nucleotide sequences are not covalently bonded either to the co-surfactant, or to the amphiphilic lipid or to any optional imaging agent. The said nucleotide sequences are solely linked to the droplets of the nanoemulsion via electrostatic interactions with the cationic surfactants. This is of great advantage since the said nucleotide sequences once released from their site of action are not denatured and can play their expected role. In addition, it is not necessary to prepare derivatives of nucleotide sequences which would be costly. Commercially available nucleotide sequences can therefore be complexed to the droplets without prior modification.

The droplets of the <<final>> formulation generally have a diameter of between 20 and 250 nm, typically between 40 and 200 nm. In particular this diameter can be measured by dynamic light scattering using Malvern ZetaSizer apparatus.

Location of the Components of the Droplets

As illustrated in FIG. 1, the droplets of the formulation according to the invention are organised in a core-ring form in which:
the core comprises:
   the solubilising lipid,
   optional oil,
   optional imaging agent,
   optional therapeutic agent if it is lipophilic,
the crown comprises:
   the amphiphilic lipid,
   cationic surfactant,
   co-surfactant (optionally grafted with a molecule of interest),
   the nucleotide sequence able to modulate endogenous mechanisms of RNA interference,
   optional helper lipid,
   optional therapeutic agent if it is amphiphilic,
   optional surfactant of formula (I).

This organisation is the same in the <<premix>> formulation, except that in this case the crown does not contain a nucleotide sequence able to modulate endogenous mechanisms of RNA interference.

### [Kit]

According to a second aspect the invention concerns a kit comprising the <<premix>> formulation such as defined above and, separately, at least one single or double strand nucleotide sequence, comprising fewer than 200 bases for a single strand nucleotide sequence or fewer than 200 base pairs for a double strand nucleotide sequence, able to modulate endogenous mechanisms of RNA (ribonucleic acid) interference.

In the kit, the <<premix>> formulation is physically separated from at least one nucleotide sequence. It is possible for example to use a kit comprising at least two containers, one for the <<premix>> formulation and at least one for a nucleotide sequence or preferably several each containing nucleotide sequences of different type.

As explained above the <<premix>> formulation, in particular by means of:
the mole proportion of cationic surfactant in the crown of the droplets,
the mole proportion of amphiphilic lipid in the crown of the droplets,
the mole proportion of co-surfactant in the crown of the droplets,
the fact that the co-surfactant comprises a poly(ethylene oxide) chain comprising at least 25 ethylene oxide units,
the presence of solubilising lipid in the core of the droplets, and/or
the mole and/or weight proportion of core in the droplets
   is advantageously stable. This stability of the <<premix>> formulation allows the long-term storage of the <<premix>> formulation, which is a prerequisite for the storing and marketing of the above-defined kit.

It is advantageously possible to prepare the <<final>> formulation from the <<premix>> formulation and the sequence just before use of the <<final>> formulation. The <<premix>> formulation can be stored. It is therefore not necessary to prepare the <<premix>> emulsion every time it is desired to use the <<final>> formulation, which amounts to an advantage in terms of savings in time and cost.

### [Preparation Method]

According to a third aspect the invention concerns the method for preparing the above-defined formulation.

Typically, the different oil constituents are first mixed to prepare an oily premix for the dispersed phase of the emulsion, and it is then dispersed in an aqueous phase under shear effect.

The preparation method typically comprises the following steps:
(i) preparing an oil phase comprising a solubilising lipid, an amphiphilic lipid, the cationic surfactant;
(ii) preparing an aqueous phase optionally comprising the co-surfactant;
(iii) dispersing the oil phase in the aqueous phase under the action of sufficient shear to form a <<premix>> formulation; then
(iv) adding nucleotide sequences able to modulate endogenous mechanisms of RNA interference to the formed <<premix>> formulation; then
(v) recovering the formulation thus formed.

Step (i)

In general the preparation of the oil phase comprises the mixing of the oily components of the formulation (solubilising lipid/amphiphilic lipid/cationic surfactant). If the formulation comprises a lipid able to facilitate cytosolic release via destabilisation of the endosomal membrane, and/or an oil, and/or an imaging agent, and/or a therapeutic agent, these are generally added to the oil phase at step (i).

Mixing can optionally be facilitated by placing one of the constituents or the complete mixture in solution in a suitable organic solvent. The organic solvent is then evaporated to obtain a homogenous oily premix for the dispersed phase.

Also, it is preferable to conduct the pre-mixing (step (i)) at a temperature at which all the ingredients are liquid.

Step (iii)

Advantageously the oil phase is dispersed in the aqueous phase in the liquid state. If one of the phases solidifies at ambient temperature, it is preferable to perform mixing by heating one or preferably both phases to melting temperature or higher.

Emulsification under shear effect is preferably performed using a sonicator or microfluidiser. Preferably the aqueous phase and then the oil phase are added in the desired proportions to a cylindrical container and the sonicator is immersed in the centre thereof and set in operation for sufficient time to obtain a nanoemulsion, most often for a few minutes.

At the end of step (iii), a homogeneous nanoemulsion is generally obtained in which:
the mean diameter of the oil droplets is generally greater than 10 nm and smaller than 200 nm, preferably between 30 and 190 nm; and
the zeta potential is higher than 20 mV, generally between 25 mV and 60 mV, preferably between 40 and 55 mV, preferably when the aqueous phase of the formulation is a 0.15 mM aqueous solution of NaCl.

The nanoemulsion obtained at the end of step (iii) corresponds to the <<premix>> formulation defined above.

Step (iv)

Step (iv) then allows the preparing of the formulation used to deliver the said nucleotide sequences, by complexing the said nucleotide sequences on the <<premix>> formulation obtained at the end of step (iii).

In general the nucleotide sequences able to modulate endogenous mechanisms of RNA interference are added to the formed nanoemulsion and the mixture obtained is mixed at ambient temperature e.g. for 30 minutes.

At step (iv), the nucleotide sequences able to modulate endogenous mechanisms of RNA interference, carrying negatively charged phosphate groups, are linked via electrostatic bonds to the droplets of which the surface is positively charged by means of the cationic groups of the cationic surfactants. Complexes are thus formed between the nucleotide sequences able to modulate endogenous mechanisms of RNA interference and the droplets of the nanoemulsion. The nucleotide sequences able to modulate endogenous mechanisms of RNA interference are generally added to the aqueous phase e.g. water free of nucleases, cell culture media, cell media optimised for transfection in particular Opti-MEM medium, or buffer solutions and in particular 4-(2-hydroxyethyl)-1-piperazine ethane sulfonic acid (HEPES). Step (iv) is typically conducted at ambient temperature (25° C.), under simple homogenisation or agitation (e.g. between 100 and 1000 rpm) for a time of between 5 minutes and 2 hours, for example in the order of 30 minutes.

Preferably at step (iv), the quantity of added nucleotide sequences is such that the ratio between the amount of positive charges due to the cationic surfactant in the <<premix>> formulation and the amount of negative charges provided by the nucleotide sequences added to the medium is higher than 8:1. It is within the reach of persons skilled in the art to calculate the amount of negative charges provided by the nucleotide sequences added to the medium and the amount of positive charges due to the cationic surfactant in the <<premix>> formulation. A said ratio allows quantitative complexing to be obtained i.e. there no longer remain in the medium any free nucleotide sequences able to modulate endogenous mechanisms of RNA interference.

Step (iv) can be monitored by various methods, for example by:
agarose gel electrophoresis which allows observation of the migration of the nucleotide sequences able to modulate endogenous mechanisms of RNA interference. With good complexing, the nucleotide sequences able to modulate endogenous mechanisms of RNA interference are heavier and can be seen in the wells. With lesser complexing, free nucleotide sequences able to modulate endogenous mechanisms of RNA interference will migrate to another position.
dynamic light scattering (DLS), by observing the impact of complexing on hydrodynamic diameter. The more complexing is efficient the more the profile tends towards monomodal distribution.

At the end of step (iv), a homogeneous nanoemulsion is generally obtained in which the mean diameter of the oil droplets is generally greater than 10 nm and smaller than 200 nm, preferably between 60 and 200 nm.

Advantageously the method for preparing the formulation does not require chemical modification of the nucleotide sequences, and in particular does not require covalent grafting thereof onto another component of the formulation. It is therefore very easy to prepare numerous formulations of the invention in parallel containing nucleotide sequences.

Optional Subsequent Steps

The formulation can be purified, for example by column purification or dialysis.

Before packaging, the emulsion can be diluted and/or sterilised, for example by filtration or dialysis. This step allows the removal of any aggregates which may have been formed during preparation of the emulsion.

The emulsion thus obtained is ready for use, optionally after dilution.

Preparation of a Formulation Comprising a Surfactant of Formula (I)

In the embodiment in which the formulation comprises a surfactant of formula (I), the nanoemulsion used for complexing with the nucleotide sequences able to modulate endogenous mechanisms of RNA interference can be prepared using a method comprising the contacting of:
an emulsion 1 comprising a continuous aqueous phase and a dispersed phase in the form of droplets comprising an amphiphilic lipid and a surfactant of following formula (LI):

L₁-X₁—H₁—Y₁-G₁ (LI),
with an emulsion 2 comprising a continuous aqueous phase and a dispersed phase in the form of droplets comprising an amphiphilic lipid and a surfactant of following formula (LII):

G₂-Y₂—H₂—X₂-L₂ (LII)
where L₁, X₁, H₁, Y₁, L₂, X₂, H₂ and Y₂ are such as defined above, and G₁ and G₂ are groups able to react to form group G such as defined above,
   under conditions allowing the reaction of the surfactants of formulas (LI) and (LII) to form the surfactant of formula (I) such as defined above, after which covalent bonds between the droplets in the dispersed phase are formed.

The continuous aqueous phases of emulsions 1 and 2 comprise a co-surfactant such as defined above. The dispersed phases of emulsions 1 and 2 comprise a solubilising lipid such as defined above. The dispersed phase of emulsion 1 and/or the dispersed phase of emulsion 2 comprises a cationic surfactant such as defined above.

When group G comprises a single G′ group, the groups G₁ and G₂ are typically groups able to react with one another to form group G.

When group G comprises several G′ groups, the emulsions 1 and 2 are generally contacted with a compound able to react with the surfactants of formulas (LI) and (LII) to form group G. This compound typically comprises at least a v number of G′₁ functions able to react with group G₁ and a w number of G′₂ functions able to react with group G₂.

Therefore in the embodiment in which the G group meets formula -G′-Y₃-G′- defined above, the method for preparing the formulation typically comprises the contacting of:
an emulsion 1 such as defined above;
and an emulsion 2 such as defined above;
with a compound of formula G′₁-Y₃-G′₂ where Y₃ is such as defined above, G′₁ is a group able to react with G₁ to form the first group G′ such as defined above and G′₂ is a group able to react with G₂ to form the second group G′ such as defined above (of same or different type to the first group G′),
   under conditions allowing reaction of the surfactants of formulas (LI) et (LII) and of the compound of formula G′₁-Y₃-G′₂ to form the surfactant of formula (I) in which group G meets formula -G′-Y₃-G′- defined above, after which covalent bonds are formed between the droplets of the dispersed phase.

Similarly in the embodiment defined above in which the G group is a dendrimer comprising (v+w) G′ groups, the method for preparing the formulation typically comprises the contacting of:
an emulsion 1 such as defined above;
an emulsion 2 such as defined above;
with a dendrimer of formula (G′₁)ᵥ—Y₄-(G′₂)_{w} in which v and w are such as defined above, G′₁ is independently a group able to react with G₁ to form a G′ group such as defined above and G′₂ is independently a group able to react with G₂ to form a group G′ such as defined above (each G′ being of same or different type to the other G′ groups) and Y₄ is the backbone of a dendrimer,
   under conditions allowing the reaction of the surfactants of formulas (LI) and (LII) and of the dendrimer of formula (G′₁)ᵥ—Y₄-(G′₂)_{w} to form the surfactant of formula (I) in which group G is a dendrimer comprising (v+w) groups G′, after which covalent bonds are formed between the droplets of the dispersed phase.
   For example, to form a group G of formula (XXX), (XXXI), (XXXII) and (XXXIII), the compound of formula (G′₁)ᵥ—Y₄-(G′₂)_{w} can respectively have one of the following formulas (XXX′), (XXXI′), (XXXII′) and (XXXIII′):

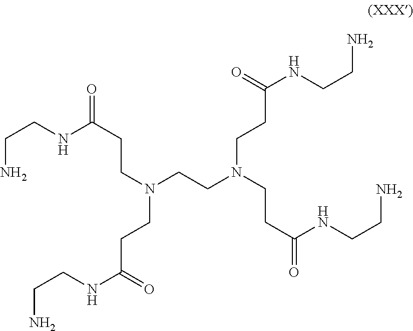 [SMILES: NCCNC(=O)CCN(CCC(=O)NCCN)CCN(CCC(=O)NCCN)CCC(=O)NCCN]

(where G′₁ and G′₂- represent NH₂ and v and w represent 2),

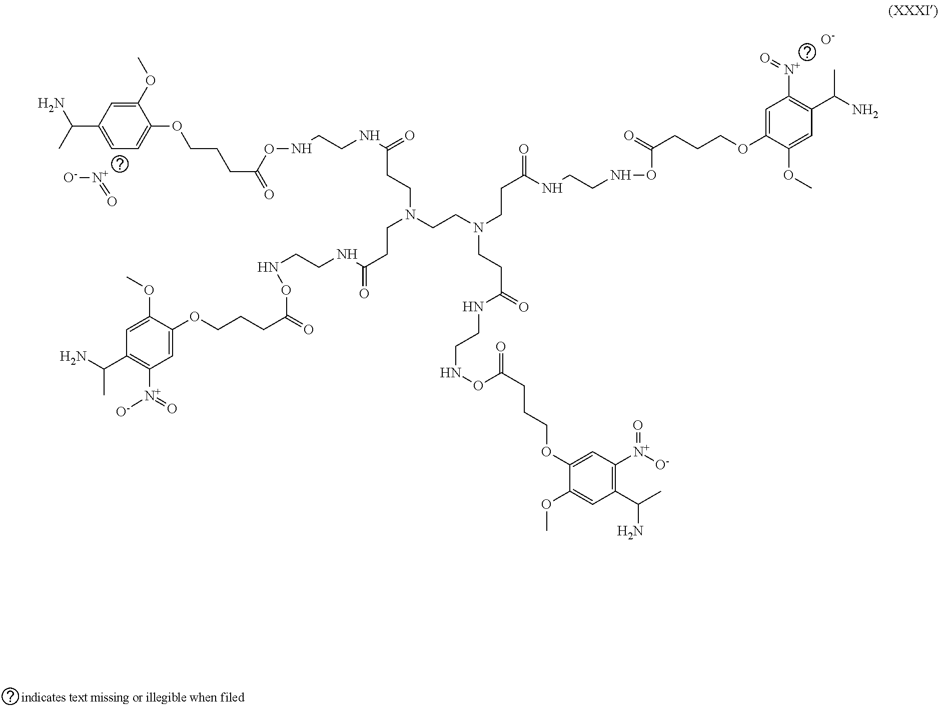

(where G′₁ and G′₂- represent NH₂ and v and w represent 2),

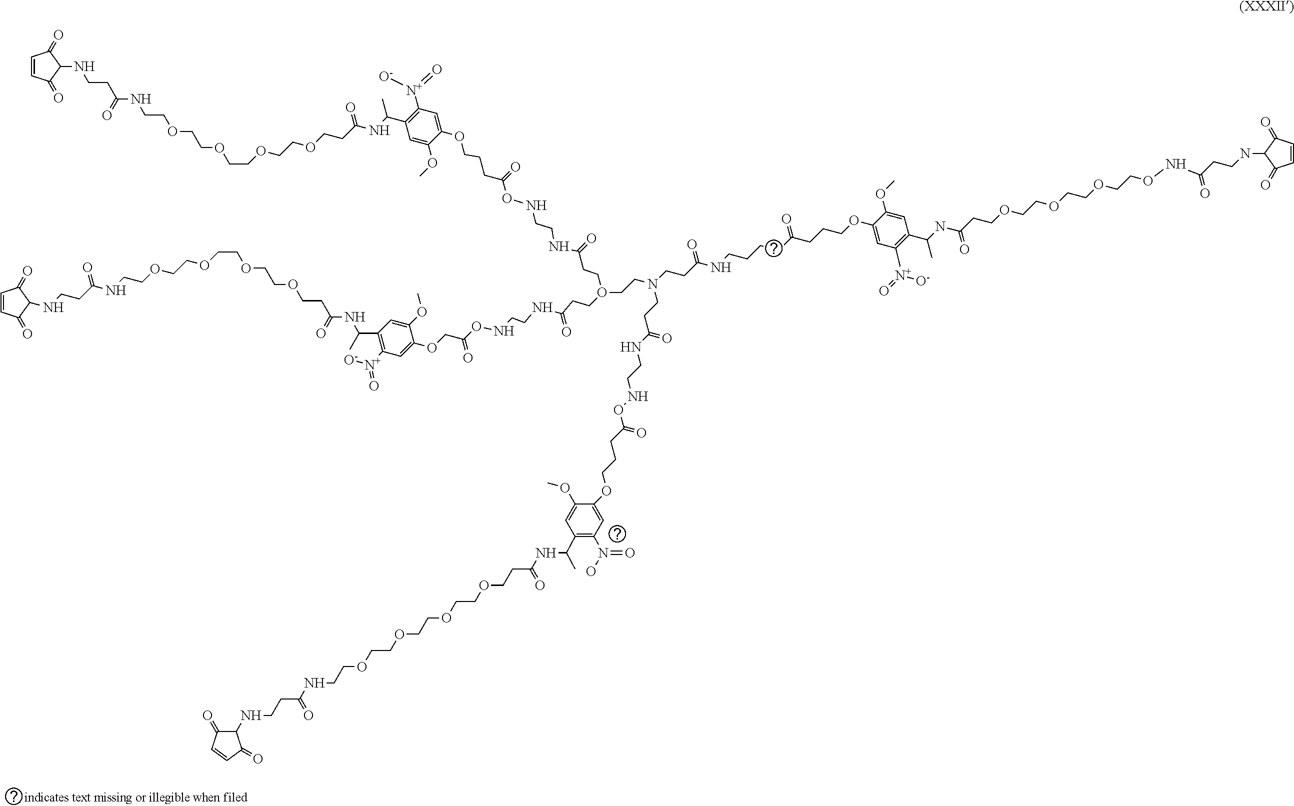

(where G′₁ and G′₂- represent

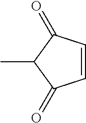

and v and w represent 2),

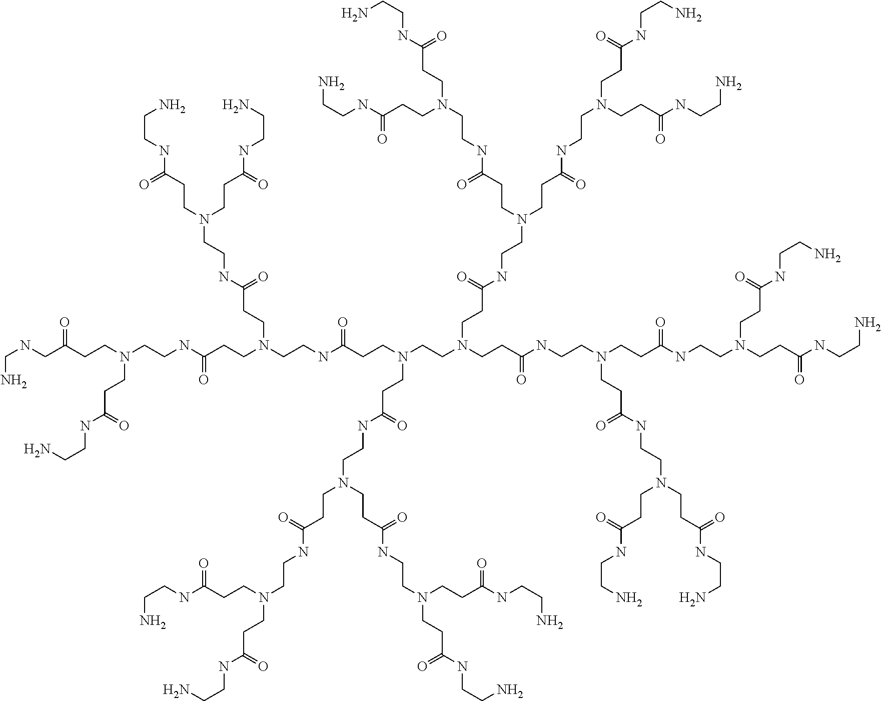 [SMILES: NCCNC(=O)CCN(CCNC(=O)CCN(CCNC(=O)CCN(CCC(=O)NCCN(CCC(=O)NCCN(CCC(=O)NCCN)CCC(=O)NCCN)CCC(=O)NCCN(CCC(=O)NCCN)CCC(=O)NCCN)CCN(CCC(=O)NCCN(CCC(=O)NCCN(CCC(=O)NCCN)CCC(=O)NCCN)CCC(=O)NCCN(CCC(=O)NCCN)CCC(=O)NCCN)CCC(=O)NCCN(CCC(=O)NCCN(CCC(=O)NCCN)CCC(=O)NCCN)CCC(=O)NCCN(CCC(=O)NCCN)CCC(=O)NCCN)CCC(=O)NCCN(CCC(=O)NCCN)CCC(=O)NCCN)CCC(=O)CNCN]

(where G′₁ and G′₂- represent NH₂ and v and w represent 8).

In the light of their general chemistry knowledge, persons skilled in the art are able to select the type of groups G′₁, G′₂, Y₃, Y₄, G₁ and G₂ to be used to form group G and the conditions allowing the reaction. The usual reactions of organic chemistry can be used in particular those described in <<Comprehensive Organic Transformations: A Guide to Functional Group Preparations>> by Richard C. Larock published by John Wiley & Sons Inc, and the references cited therein. Therefore the examples of groups G₁ and G₂ below are given by way of illustration and are non-limiting.

Typically when group G is formed of one G′ group, the groups G₁ and G₂ of the compounds of formulas (LI) and (LII) can be chosen as follows for example:
G₁ is a thiol (—SH) and G₂ is:
   either a maleimide, a surfactant of formula (I) then being formed in which G comprises a G′ group representing a group of formula (XIV) where A₁₀₂ represents N;
   or a vinylsulfone, a surfactant of formula (I) then being formed in which G comprises a G′ group representing a group of formula (XVI);
   or a —S—S-pyridinyl or —SH group, a surfactant of formula (I) then being formed in which G comprises a G′ group representing a group of formula (XV);
G₁ is a hydroxyl and G₂ is —COOH or an activated ester, a surfactant of formula (I) then being formed in which G comprises a group G′ representing a group of formula (XXIII);
G₁ is an amine —NH₂ and G₂ is —COOH or an activated ester, a surfactant of formula (I) then being formed in which G comprises a group G′ representing a group of formula (XI);
G₁ is a hydroxyl and G₂ is an activated carbonate, a surfactant of formula (I) then being formed in which G comprises a group G′ representing a group of formula (XIX);
G₁ is an amine —NH₂ and G₂ is an activated carbonate, a surfactant of formula (I) then being formed in which G comprises a group G′ representing a group of formula (XII);
G₁ is an amine —NH₂ and G₂ is a —CHO aldehyde, a surfactant of formula (I) then being formed in which G comprises a group G′ representing a group of formula (XXI);
G₁ is a hydrazide of formula —(C═O)—NH—NH₂ and G₂ is a —(C═O)—R10₂ group, a surfactant of formula (I) then being formed in which G comprises a group G′ representing a group of formula (XIII);
G₁ is an alkyne and G₂ is an azide, a surfactant of formula (I) then being formed in which G comprises a group G′ representing a group of formula (XVIII);
G₁ is a cyclooctyne and G₂ is an azide, a surfactant of formula (I) then being formed in which G comprises a group G′ representing a group of formula (XVIII′);
G₁ is a furan and G₂ is a maleimide, a surfactant of formula (I) then being formed in which G comprises a group G′ representing a group of formula (XVII);
G₁ is an aldehyde and G₂ is an amine, a surfactant of formula (I) then being formed in which G comprises a group G′ representing a group of formula (XXI);

G₁ is a phosphate of formula —O—P(═O)(OH)₂ and G₂ is a hydroxyl, a surfactant of formula (I) then being formed in which G comprises a group G′ representing a group of formula (XXII);
G₁ is a good leaving group LG and G₂ is a group of following formula

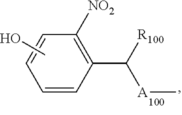

a surfactant of formula (I) then being formed in which G comprises a group G′ representing a group of formula (XXIV) in which A₁₀₁ is O;
G₁ is a hydroxyl or —NH₂ amine and G₂ represents a group of following formula

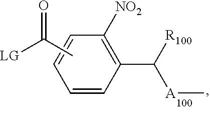

a surfactant of formula (I) then being formed in which G comprises a group G′ representing a group of formula (XXIV) in which A₁₀₁ respectively represents —O—(CO)— or —NH—(CO);
G₁ is a good leaving group LG and G₂ is a hydroxyl, a surfactant of formula (I) then being formed in which G comprises a G′ group representing a group of formula (XXV);
G₁ is a good leaving group LG and G₂ is an amine —NH₂, a surfactant of formula (I) then being formed in which G comprises a group G′ representing a group of formula (XXVI);

G₁ is an oxyamine —O—NH₂ and G₂ is an aldehyde, a surfactant of formula (I) then being formed in which G comprises a group G′ representing a group of formula (XXVII).
When group G comprises several groups G′, the choice of groups reacting together: G′₁ and G₁, G′₂ and G₂ can be made in the same manner by replacing groups G₁ or G₂ in the above-mentioned examples with G′₁ or G′₂.

The emulsions 1 and 2 used in the method can be prepared using the first method described above comprising steps (i), (ii), (iii) and (v) (and not performing step (iv) to add the nucleotide sequences able to modulate endogenous mechanisms of RNA interference).

### [Use]

The formulation is advantageously stable and scarcely toxic. It can be used as transfecting agent and/or as system to deliver nucleotide sequences able to modulate endogenous mechanisms of RNA interference.

If the formulation comprises an imaging agent, it is advantageously possible to monitor the delivery of the nucleotide sequences able to modulate endogenous mechanisms of RNA interference and/or cell transfection using fluorescence tracking for example if the imaging agent is a lipophilic fluorophore.

As Transfecting Agent

According to a fourth aspect, the invention concerns a method for inserting nucleotide sequences able to modulate endogenous mechanisms of RNA interference into a eukaryote cell, comprising the contacting of the eukaryote cell with the formulation of the invention. This method is a transfection method.

The insertion method can be performed in vitro. In this case and in general the contacting of the eukaryote cell with the formulation of the invention takes place in a buffer solution e.g. OptiMEM® medium. The contacting generally lasts between 8 and 96 hours, typically in the order of 72 hours at a temperature in the order of 37° C.

In general after the contacting step the cells are recovered.

Not only do the droplets of the formulation of the invention allow efficient transfection of cells but in addition they are functionally active and allow silencing of the expression of the gene of interest.

Without wishing to be bound by any particular theory, it would seem that once the droplets of the nanoemulsion of the invention have transfected the cell, endosomal escape releases the droplets and the nucleotide sequences able to modulate endogenous mechanisms of RNA interference in the cytoplasm, and hence at the site of action of the nucleotide sequences able to modulate endogenous mechanisms of RNA interference, which apparently prevents degradation of these nucleotide sequences in the lysosome, enabling them to interfere with the target mRNA and thereby silence expression of the gene of interest.

In one embodiment, the formulation comprises a co-surfactant grafted by a targeting biological ligand. This embodiment advantageously allows the inserting of nucleotide sequences able to modulate endogenous mechanisms of RNA interference in eukaryote cells that are usually resistant to transfection i.e. known to be difficult to transfect, in particular stem cells, primary cells (e.g. human fibroblasts or human endothelial cells), or lymphocyte cell lines (e.g. of Jurkat type) or neuroblastomas (e.g. of SK—N—SH type). The targeting biological ligand allows facilitated transfection of cells usually resistant to transfection with the formulation.

For the Delivery of Nucleotide Sequences Able to Modulate Endogenous Mechanisms of RNA Interference

The formulation of the invention is a system for the in vitro or in vivo delivery of nucleotide sequences able to modulate endogenous mechanisms of RNA interference.

Therefore, according to a fifth aspect, the invention concerns the above-defined formulation for use thereof in the prevention and/or treatment of a disease.

The disease is typically a disease for which the silencing of one or more genes is sought, such as:
ocular diseases, in particular age-related macular degeneration (AMD), glaucoma or congenital Pachyonychia;
cancer, solid tumours, metastases, but also chronic myeloid leukaemia;
renal disorders in particular after transplantation or acute renal deficiency;
hypercholesterolemia;
viral diseases related in particular to infection by hepatitis C virus (HCV), by human immunodeficiency virus (HIV) and by respiratory syncytial virus (RSV).

Advantageously as explained above, the formulation of the invention protects the nucleotide sequences able to modulate endogenous mechanisms of RNA interference vis-à-vis a patient's body (and proteins) to which the formulation is administered (in particular by preventing early metabolism thereof).

The formulation may comprise a therapeutic agent allowing treatment of disease, which provides the benefit of a double therapeutic effect; the effect induced by the nucleotide sequences able to modulate endogenous mechanisms of RNA interference and the effect induced by the therapeutic agent.

Since it can be prepared exclusively from constituents approved for use in man, it is of interest for administration via parenteral route. However it is also possible to envisage administration via other routes, in particular via oral route, via topical route or via ophthalmologic route.

A method for the prevention and/or treatment of a disease comprising the administration to a mammal, preferably a human in need thereof, of an efficient quantity of the formulation such as defined above is also one of the subjects of the present invention.

### DEFINITIONS

In the present application, by the term <<nanoemulsion>> is meant a composition having at least two phases, in general an oil phase and an aqueous phase in which the mean size of the dispersed phase is smaller than 1 micron, preferably 10 to 500 nm and in particular 20 to 200 nm, and most preferably 50 to 200 nm (see article by C. Solans, P. Izquierdo, J. Nolla, N. Azemar and M. J. Garcia-Celma, Curr Opin Colloid In, 2005, 10, 102-110).

In the meaning of the present application, the expression <<dispersed phase>> designates the droplets comprising the optional oil/the cationic surfactant(s)/the solubilising lipid/the amphiphilic lipid/the co-surfactant/optional surfactant of formula (I)/optional helper lipid/optional imaging agent/optional therapeutic agent/optional nucleotide sequences able to modulate endogenous mechanisms of RNA interference. The dispersed phase is generally free of aqueous phase.

The term <<droplet>> encompasses both the droplets of liquid oil properly so-called and the solid particles derived from emulsions of oil-in-water type in which the dispersed phase is solid. In this latter case, the term solid emulsion is also often used.

The term <<lipid>> in this description designates all fatty bodies or substances containing fatty acids present in the fats of animal origin and in vegetable oils. They are hydrophobic or amphiphilic molecules chiefly formed of carbon, hydrogen and oxygen and having a density lower than that of water. The lipids may be in the solid state at ambient temperature (25° C.) as in waxes, or liquid state as in oils.

The term <<phospholipid>> concerns lipids having a phosphate group, in particular phosphoglycerides. Most often the phospholipids comprise a hydrophilic end formed by the optionally substituted phosphate group and two hydrophobic ends formed by fatty acid chains. Amongst the phospholipids, particular mention is made of phosphatidylcholine, phosphatidyl ethanolamine, phophatidyl, inositol, phosphatidyl serine and sphingomyeline.

The term <<lecithin>> designates phosphatidylcholine i.e. a lipid formed from a choline, a phosphate, a glycerol and from two fatty acids. It more broadly covers living phospholipid extracts of vegetable or animal origin, provided that they are mostly formed of phosphatidylcholine. These lecithins generally form mixtures of lecithins carrying different fatty acids.

The term <<fatty acid>> designates aliphatic carboxylic acids having a carbon chain of at least 4 carbon atoms. Natural fatty acids have a carbon chain of 4 to 28 carbon atoms (generally an even number). The term long chain fatty acid is used for a length of 14 to 22 carbons and very long chain if there are more than 22 carbons.

By the term <<surfactant>> is meant compounds with amphiphilic structure imparting particular affinity thereto for interfaces of oil/water and water/oil type providing them with the capability of reducing the free energy of these interfaces and stabilising dispersed systems.

By the term <<co-surfactant>> is meant a surfactant acting in addition to a surfactant to further reduce interface energy.

By the term <<hydrocarbon chain>> it is meant to designate a chain composed of carbon and hydrogen atoms, saturated or unsaturated (double or triple bond). The preferred hydrocarbon chains are the alkyls or alkenyls.

By the term <<alkylene>> it is meant to designate an aliphatic divalent hydrocarbon group, saturated, linear chain or branched, preferably linear chain.

By <<cyclic radical>> is meant a radical derived from a crown system. For example a phenylene radical is a divalent radical derived from a benzene group. By <<crown system>> is meant a carbocycle or heterocycle, saturated, unsaturated or aromatic (aryl or heteroaryl).
a carbocycle: a saturated crown composed of carbon atoms (the preferred saturated carbocycles in particular being a cycloalkyl, such as a cyclopentyl or cyclohexyl), unsaturated (e.g. a cyclohexene) or aromatic (i.e. a phenyl);
a heterocycle: a crown group comprising, unless indicated otherwise, 5 to 6 atoms and comprising one or more heteroatoms selected from among O, N and/or S. The said heterocycle can be saturated or partly unsaturated and may comprise one or more double bonds. In this case the term heterocycloalkyl group is used. It may also be aromatic comprising, unless indicated otherwise, 5 to 6 atoms and then represent a heteroaryl group.
   as non-aromatic heterocycle or heterocycloalky the following can be cited: pyrazolidinyl, imidazolidinyl, tetrahydrothiophenyl, dithiolanyl, thiazolidinyl, tetrahydropyranyl, Idioxanyl, morpholinyl, piperidyl, piperazinyl, tetrahydrothiopyranyl, thiomorpholinyl, dihydrofuranyl, 2-imidazolinyl, 2,-3-pyrrolinyl, pyrazolinyl, dihydrothiophenyl, dihydropyranyl, pyranyl, tetrahydropyridyl, dihydropyridyl, tetrahydropyrinidinyl, dihydrothiopyranyl, isoxazolidinyl,
   as heteroaryl particular mention can be made of the following representative groups: furyl, imidazolyl, isoxazolyl, isothiazolyl, oxadiazolyl, 1,2,4-oxadiazolyl, 1,3,4-oxadiazolyl, pyrazinyl, pyridazinyl, pyrazolyl, pyridyl, pyrimidinyl, pyrrolyl, 1,3,4-thiadiazolyl, 1,2,3-thiadiazolyl, thiazolyl, thienyl, triazolyl, 1,2,3-triazolyl and 1,2,4-triazolyl.

By <<activated ester>> is meant a group of formula —CO-LG, and by <<activated carbonate>> is meant a group of formula —O—CO-LG where LG is a good leaving group selected in particular from among a chlorine, imidazolyl, pentafluorophenolate, pentachlorophenolate, 2,4,5-trichlorophenolate 2,4,6-trichlorophenolate, or from among a group —O-succinimidyl, —O-benzotriazolyl, —O-(7-aza-benzotriazolyl) and —O-(4-nitrophenyl).

The invention is descried in better detail with reference to the appended Figures and following examples.

### FIGURES

FIG. 1 gives a schematic illustrating the core/ring structure of a droplet of a formulation according to the invention (<<final>> formulation with a nucleotide sequence able to modulate endogenous mechanisms of RNA interference). 1 represents the continuous aqueous phase, 2 the crown (in part) of the droplet and 3 the core (in part) of the droplet. In the crown 2 are illustrated: the amphiphilic liquid (e.g. neutral phospholipid such as lecithin), the cationic surfactant (e.g. cationic phospholipid DOTAP), the co-surfactant of which the folding poly(ethylene oxide) chain is represented in the aqueous phase and the double strand nucleotide sequence able to modulate endogenous mechanisms of RNA interference (e.g. siRNA).

FIG. 2 shows an electrophoresis with UV detection after complexing of siRNA with the formulation B10 and the concentrations specified in Table 8. The scale and siRNA (reference) are given on the left.

FIG. 3 gives the results of the amount of siRNA (ng) obtained when processing electrophoresis data with ImageJ software for the electrophoresis gel with UV detection in FIG. 2.

FIG. 4 gives the intensity obtained on ZetaSizer Malvern apparatus as a function of hydrodynamic diameter in nm for free siRNA (comparison) and for three formulations of the invention obtained by complexing with a ratio N/P: between the amount of positive charges due to the cationic surfactant in the <<premix>> formulation (due to charged nitrogen hence the N in N/P), and the amount of negative charges provided by the siRNAs (due to the phosphorus of siRNA hence the P in N/P) of 4:1, 8:1 or 16:1.

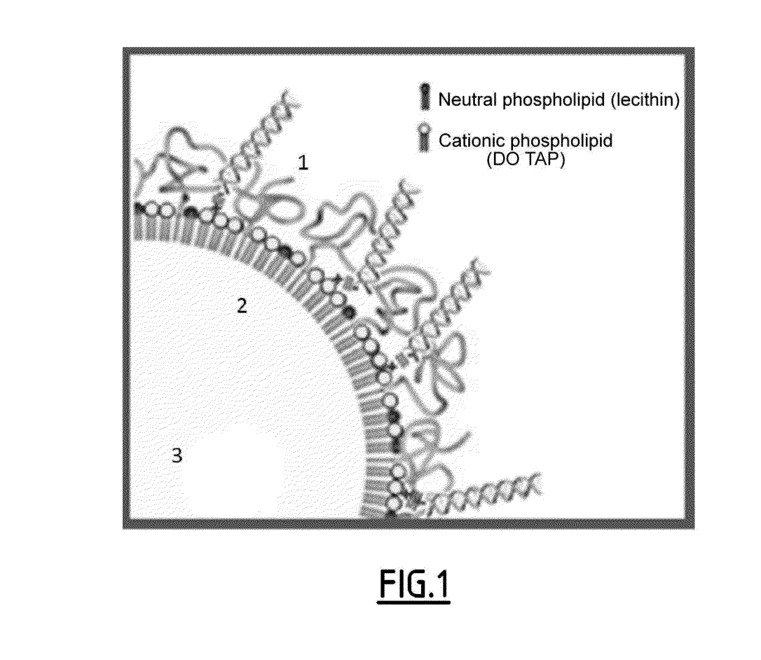

FIG. 5 shows an electrophoresis gel after UV detection with from left to right:
the scale,
free siRNA (reference)
   then the migrations obtained by siRNA complexing:
with formulation B1 (comparative example) (no complexing, the siRNA are free);
with formulation B6 with a ratio between the amount of positive charges due to the cationic surfactant in the <<premix>> formulation and the amount of negative charges provided by the siRNA of 8:1 (quantitative complexing).
with formulation B6 and a ratio between amount positive charges due to the cationic surfactant in the <<premix>> formulation and amount of negative charges provided by the siRNA of 8:1 (quantitative complexing);
with formulation B10 and a ratio between amount of positive charges due to the cationic surfactant in the <<premix>> formulation and amount of negative charges provided by the siRNA of 8:1 (quantitative complexing),
with lipofectamine (comparative example) (incomplete complexing).

FIG. 6 shows an electrophoresis gel after UV detection of a siRNA/B10 formulation complex just after complexing (T0) then 15 min, 45 min, 1 h 30, 3 h and 6 h after complexing. The scale and siRNA (reference) are on the left.

FIG. 7 gives the decrease in FITC fluorescence intensity as a % when transfecting the cell lines with:
Lipofectamine RNAimax (commercial agent);
siRNA/formulation B1 complex;
siRNA/formulation B6 complex, the formulation having been stored for 7 months at ambient temperature prior to complexing;
siRNA/formulation B6 complex, the formulation having been stored for 12 months at ambient temperature prior to complexing;
siRNA/formulation B10 complex, the formulation having been stored for 12 months at ambient temperature prior to complexing;
complex of siRNA/formulation of cationic liposomes comprising DOTAP (58 wt. %), DOPE (18 wt. %), cholesterol (2 wt. %) and DSPE-PEG3000 (22 wt. %)) (comparative).

FIG. 8 gives the fluorescence intensity (FITC) for 3 cell lines experimentally overexpressing the fluorescent protein GFP (green fluorescent protein): U2OS, PC3 and Hela for the siRNA/formulation B10 complex, the siRNA called siGFP being targeted against the mRNA encoding the GFP protein, and for the negative reference (cells incubated with a B10 formulation complexed with a negative reference siRNA i.e. <<inert>> without effect on the transcriptome of the cell called siAllStar).

FIG. 9 shows an electrophoresis gel with UV detection after complexing of synthetic microRNA of mimic type with the A3 formulation at the concentrations specified in Table 9. The scale and the microRNA Mimic (reference) and the non-complexed A3 premix formulation are on the left. The final formulations derived from complexing the A3 premix formulation and the Mimics with five different N/P ratios are given on the right.

### EXAMPLES

### Preparation and Composition of <<Premix>> Formulations Before Complexing with siRNA

The aqueous phase used was a PBS 1× buffer solution.
The suppliers of the compounds were the following:

### Lipoid S75-3: Lipoid

### Lipoid S75: Lipoid

### Lipoid S100-3: Lipoid

### DOTAP: Avanti Polar

### DOPE: Avanti Polar

### MyrjS40: Croda

Suppocire NB: Gattefossé

### Soybean oil: Croda

The hydrodynamic diameter of the droplets in the formulations and their zeta potential were measured by dynamic light scattering using ZetaSizer apparatus, Malvern. The hydrodynamic diameter of the droplets was measured in 0.1× solution of PBS, the zeta potential in 0.15 mM aqueous solution of NaCl.
Sixteen different formulations were prepared, the compositions of which are given in Tables 1 to 5.

TABLE 1

| | | A1 | | |
|---|---|---|---|---|
| | | (comp.) | A2 | A3 |
| RING | Amphiphilic lipid: | S75-3 | S75-3 | S75-3 |
| | Lipoid | | | |
| | wt. % Lipoid/droplet | 35.29 | 8.83 | 3.53 |
| | wt. % Lipoid/ring | 100 | 25 | 10 |
| | without PEG | | | |
| | wt. % Lipoid/crown | 46.15 | 11.54 | 4.62 |
| | mol. % Lipoid/crown | 70.02 | 16.86 | 6.74 |
| | Cationic surfactant | X | DOTAP | DOTAP |
| | DOTAP | | | |
| | wt. % DOTAP/droplet | 0 | 26.47 | 26.47 |
| | wt. % DOTAP/crown | 0 | 75 | 75 |
| | without PEG | | | |
| | wt. % DOTAP/crown | 0 | 34.61 | 34.61 |
| | mol. % DOTAP/crown | 0 | 54.28 | 54.24 |
| | Co-surfactant | Myrj S40 | Myrj S40 | Myrj S40 |
| | wt. % co-surfactant/ | 41.17 | 41.17 | 41.17 |
| | droplet | | | |
| | wt. % (co-surfactant)/ | 53.85 | 53.85 | 53.85 |
| | crown | | | |
| | mol. % (co-surfactant)/ | 29.98 | 28.86 | 28.84 |
| | crown | | | |
| | Helper lipid DOPE | X | X | DOPE |
| | wt. % DOPE/droplet | 0 | 0 | 5.29 |
| | wt. % DOPE/crown | 0 | 0 | 15 |
| | without PEG | | | |
| | wt. % DOPE/crown | 0 | 0 | 6.92 |
| | mol. % DOPE/crown | 0 | 0 | 10.18 |
| CORE | Solubilising lipid | Suppocire | Suppocire | Suppocire |
| | | NB | NB | NB |
| | wt. % solubilising | 75 | 75 | 75 |
| | lipid/core | | | |
| | wt. % solubilising | 17.65 | 17.65 | 17.65 |
| | lipid/droplet | | | |
| | Oil | Soybean | Soybean | Soybean |
| | | oil | oil | oil |
| | wt. % oil/core | 25 | 25 | 25 |
| | wt. % oil/droplet | 5.88 | 5.88 | 5.88 |
| | wt. % core/droplet | | 23.53 | 23.53 |
| | mol. % core/droplet | | 33.35 | 33.35 |
| | Formulation (number of | 3 | 4 | 3 |
| | repeats) | | | |
| RESULT | Hydrodynamic diameter | 124.9 | 49.1 | 44.48 |
| | (nm) - average | | | |
| | ZP (mV)_in 0.15 mM | −26 | 25.38 | 30.87 |
| | NaCl | | | |
| | Stability | ok | ok | ok |
| | Complexing (%) | 0 | 100 | 100 |
| | Silencing efficacy (%) | 0 | 72.88 | 75.06 |

TABLE 2

| | | B1 | | |
|---|---|---|---|---|
| | | (comp.) | B2 | B3 |
| CORE | Amphiphilic lipid: | S75-3 | S75 | S100-3 |
| | Lipoid | | | |
| | wt. % Lipoid/droplet | 8.75 | 4.25 | 4.25 |
| | wt. % Lipoid/crown | 100 | 50 | 50 |
| | without PEG | | | |
| | wt. % Lipoid/crown | 15.98 | 7.99 | 7.99 |
| | mol. % Lipoid/crown | 34.14 | 17.89 | 17.89 |
| | Cationic surfactant | X | DOTAP | DOTAP |
| | DOTAP | | | |
| | wt. % DOTAP/droplet | 0 | 4.25 | 4.25 |
| | wt. % DOTAP/crown | 0 | 50 | 50 |
| | without PEG | | | |
| | wt. % DOTAP/crown | 0 | 7.99 | 7.99 |
| | mol. % DOTAP/crown | 0 | 17.85 | 17.85 |
| | Co-surfactant | Myrj S40 | Myrj S40 | Myrj S40 |
| | wt. % co-surfactant/ | 46 | 46 | 46 |
| | droplet | | | |
| | wt. %(co-surfactant)/ | 84.02 | 84.02 | 84.02 |
| | crown | | | |
| | mol. % (co-surfactant)/ | 65.86 | 64.27 | 64.27 |
| | crown | | | |
| | Helper lipid DOPE | X | X | X |
| | wt. % DOPE/droplet | 0 | 0 | 0 |
| | wt. % DOPE/crown | 0 | 0 | 0 |
| | without PEG | | | |
| | wt. % DOPE/crown | 0 | 0 | 0 |
| | mol. % DOPE/crown | 0 | 0 | 0 |
| CORE | Solubilising lipid | Suppocire | Suppocire | Suppocire |
| | | NB | NB | NB |
| | wt. % solubilising | 75 | 75 | 75 |
| | lipid/core | | | |
| | wt. % solubilising | 33.94 | 33.94 | 33.94 |
| | lipid/droplet | | | |
| | Oil | Soybean | Soybean | Soybean |
| | | oil | oil | oil |
| | wt. % oil/core | 25 | 25 | 25 |
| | wt. % oil/droplet | 11.31 | 11.31 | 11.31 |
| | wt. % core/droplet | | 45.25 | 45.25 |
| | mol. % core/droplet | | | |
| | Formulation (number of | 6 | 2 | 2 |
| | repeats) | | | |
| RESULT | Hydrodynamic diameter | 59.51 | 42.11 | 6..43 |
| | (nm) - average | | | |
| | ZP (mV)_in 0.15 mM | −21.8 | 21.4 | 6.72 |
| | NaCl | | | |
| | Stability | ok | ok | ok |
| | Complexing (%) | 0 | ND | ND |
| | Silencing efficacy (%) | 0 | 0 | 0 |

TABLE 3

| | | B4 | | |
|---|---|---|---|---|
| | | (comp.) | B5 | B6 |
| RING | Amphiphilic lipid: | S75-3 | S75-3 | S75-3 |
| | Lipoid | | | |
| | wt. % Lipoid/droplet | 6.58 | 2.19 | 2.84 |
| | wt. % Lipoid/crown | 100 | 25 | 25 |
| | without PEG | | | |
| | wt. % Lipoid/crown | 14.29 | 4 | 6.9 |
| | mol. % Lipoid/crown | 31.24 | 8.39 | 12.39 |
| | Cationic surfactant | X | DOTAP | DOTAP |
| | DOTAP | | | |
| | wt. % DOTAP/droplet | 0 | 6.56 | 8.52 |
| | wt. % DOTAP/crown | 0 | 75 | 75 |
| | without PEG | | | |
| | wt. % DOTAP/crown | 0 | 11.99 | 20.67 |
| | mol. % DOTAP/crown | 0 | 26.99 | 39.87 |
| | Co-surfactant | Myrj S40 | Myrj S40 | Myrj S40 |
| | wt. % co-surfactant/ | 39.48 | 46 | 29.87 |
| | droplet | | | |
| | wt. % (co-surfactant)/ | 85.71 | 84.02 | 72.43 |
| | crown | | | |
| | mol. % (co-surfactant)/ | 68.76 | 64.63 | 47.74 |
| | crown | | | |
| | Helper lipid DOPE | X | X | X |
| | wt. % DOPE/droplet | 0 | 0 | 0 |
| | wt. % DOPE/crown | 0 | 0 | 0 |
| | without PEG | | | |
| | wt. % DOPE/crown | 0 | 0 | 0 |
| | mol. % DOPE/crown | 0 | 0 | 0 |
| CORE | Solubilising lipid | Suppocire | Suppocire | Suppocire |
| | | NB | NB | NB |
| | wt. % solubilising | 75 | 75 | 75 |
| | lipid/core | | | |
| | wt. % solubilising | 40.46 | 33.94 | 44.07 |
| | lipid/droplet | | | |
| | Oil | Soybean | Soybean | Soybean |
| | | oil | oil | oil |
| | wt. % oil/core | 25 | 25 | 25 |
| | wt. % oil/droplet | 13.49 | 11.31 | 14.69 |
| | wt. % core/droplet | | 45.25 | 58.76 |
| | mol. % core/droplet | | | 73.99 |
| | Formulation (number of | 3 | 4 | 6 |
| | repeats)) | | | |
| RESULT | Hydrodynamic diameter | 84.88 | 56.68 | 86.77 |
| | (nm) - average | | | |
| | ZP (mV)_in 0.15 mM | −18.89 | 26.51 | 36.38 |
| | NaCl | | | |
| | Stability | ok | ok | ok |
| | Complexing (%) | 0 | 100 | 100 |
| | Silencing efficacy (%) | 0 | 0 | 42.81 |

TABLE 4

| | | B9 (Comp.) | B10 |
|---|---|---|---|
| RING | Amphiphilic lipid: Lipoid | S75-3 | S75-3 |
| | wt. % Lipoid/droplet | 0 | 1.71 |
| | wt. % Lipoid/crown without | 0 | 15 |
| | PEG | | |
| | wt. % Lipoid/crown | 0 | 4.14 |
| | mol. % Lipoid/crown | 0 | 7.43 |
| | Cationic surfactant DOTAP | DOTAP | DOTAP |
| | wt % DOTAP/droplet | 8.25 | 8.25 |
| | wt. % DOTAP/crown without | 75 | 75 |
| | PEG | | |
| | wt. % DOTAP/crown | 20.67 | 20.67 |
| | mol. % DOTAP/crown | 39.87 | 39.87 |
| | Co-surfactant | Myrj S40 | Myrj S40 |
| | wt % co-surfactant/droplet | 29.87 | 29.87 |
| | wt. % (co-surfactant)/crown | 72,.43 | 72.43 |
| | mol. % (co-surfactant/crown | 47.74 | 47.74 |
| | Helper lipid DOPE | DOPE | DOPE |
| | wt. % DOPE/droplet | 2.84 | 1.71 |
| | wt. % DOPE/crown without | 25 | 10 |
| | PEG | | |
| | wt. % DOPE/crown | 6.9 | 2.76 |
| | mol. % DOPE/crown | 10.18 | 4.99 |
| CORE | Solubilising lipid | Suppocire | Suppocire |
| | | NB | NB |
| | wt. % solubilising lipid/ | 75 | 75 |
| | core | | |
| | wt. % solubilising lipid/ | 44.07 | 44.07 |
| | droplet | | |
| | Oil | Soybean | Soybean |
| | | oil | oil |
| | wt. % oil/core | 25 | 25 |
| | wt. % oil/droplet | 14.69 | 14.69 |
| | wt. % core/droplet | | 58.76 |
| | mol. % core/droplet | | 73.74 |
| | Formulation (number of | 1 poor | 5 |
| | repeats) | formulation | |
| RESULT | Hydrodynamic diameter | ND | 88.64 |
| | (nm) - average | | |
| | ZP (mV)_in 0.15 mM NaCl | ND | 36.9 |
| | Stability | ND | Ok |
| | Complexing (%) | ND | 100 |
| | Silencing efficacy (%) | ND | 49.34 |

TABLE 5

| | | C1 | | |
|---|---|---|---|---|
| | | (comp.) | C2 | C3 |
| RING | Amphiphilic lipid: | S75-3 | S75-3 | S75-3 |
| | Lipoid | | | |
| | wt. % Lipoid/droplet | 28.44 | 7.11 | 4.27 |
| | wt. % Lipoid/crown | 100 | 25 | 15 |
| | without PEG | | | |
| | wt. % Lipoid/crown | 70.24 | 17.56 | 10.54 |
| | mol. % mol. Lipoid/ | 86.55 | 20.65 | 12.38 |
| | crown | | | |
| | Cationic surfactant | X | DOTAP | DOTAP |
| | DOTAP | | | |
| | wt. % DOTAP/droplet | 0 | 21.33 | 21.33 |
| | wt. % DOTAP/crown | 0 | 75 | 75 |
| | without PEG | | | |
| | wt. % DOTAP/crown | 0 | 52.68 | 52.68 |
| | mol. % DOTAP/crown | 0 | 66.51 | 66.47 |
| | Co-surfactant | Myrj S40 | Myrj S40 | Myrj S40 |
| | wt. % co-surfactant/ | 12.05 | 12.05 | 12.05 |
| | droplet | | | |
| | wt. % (co-surfactant/ | 29.76 | 29.76 | 29.76 |
| | crown | | | |
| | mol. % (co-surfactant/ | 13.45 | 12.84 | 12.83 |
| | crown | | | |
| | Helper lipid DOPE | X | X | DOPE |
| | wt. % DOPE/droplet | 0 | 0 | 2.84 |
| | wt. % DOPE/crown | 0 | 0 | 10 |
| | without PEG | | | |
| | wt. % DOPE/crown | 0 | 0 | 7.02 |
| | mol. % DOPE/crown | 0 | 0 | 8.32 |
| CORE | Solubilising lipid | Suppocire | Suppocire | Suppocire |
| | | NB | NB | NB |
| | wt. % solubilising | 75 | 75 | 75 |
| | lipid/core | | | |
| | wt. % solubilising | 44.63 | 44.63 | 44.63 |
| | lipid/droplet | | | |
| | Oil | Soybean | Soybean | Soybean |
| | | oil | oil | oil |
| | wt. % oil/core | 25 | 25 | 25 |
| | wt. % oil/droplet | 14.88 | 14.88 | 14.88 |
| | wt. % core/droplet | | 59.51 | 59.51 |
| | mol. % core/droplet | | 65.85 | |
| | Formulation (number of | 4 | 4 | 3 |
| | repeats) | | | |
| RESULT | Hydrodynamic diameter | 153.03 | 162.2 | 168.9 |
| | (nm) - average | | | |
| | ZP (mV)_in 0.15 mM | −37.71 | 53.7 | 51.83 |
| | NaCl | | | |
| | Stability | ok | ok | ok |
| | Complexing (%) | 0 | 100 | 100 |
| | Silencing efficacy (%) | 0 | 80.51 | 81.72 |

### In Tables 1 to 5:

wt % corresponds to weight percent
mol. % corresponds to mole percent.
ND (non-determined) means that the experiment was not performed.
The percentages <</droplet>> represent percentages relative to the whole (Lipoid/DOTAP/Myrj S40/optional DOPE/Suppocire NB/Soybean oil).
The percentages <</ring>> represent percentages relative to the whole (Lipoid/DOTAP/Myrj S40/optional DOPE).
The percentages <</ring without PEG>> represent percentages relative to the whole (Lipoid/DOTAP/optional DOPE).
The percentages <</core>> represent percentages relative to the whole (Suppocire NB/Soybean oil).
Lipoid S75-3 comprises 65-75% of phosphatidylcholine. The aliphatic chains of the phospholipids are mostly saturated (mean composition: 12-16% of C16:0, 80-85% of C18:0, <5% of C18:1, <2% of C18:2).
Lipoid S75 comprises 65-75% of phosphatidylcholine. The aliphatic chains of the phospholipids are mostly unsaturated (mean composition: 17-20% of C16:0, 2-5% of C18:0, 8-12% of C18:1, 58-65% of C18:2, 4-6% of C18:3).
Lipoid S100-3 comprises >94% of phosphatidylcholine, i.e. The aliphatic chains of the phospholipids are mostly saturated (mean composition: 12-16% of C16:0, 85-88% of C18:0, <2% of C18:1, <1% of C18:2).
The formulations A1, B1, B4 and C1 are comparative examples, since they do not comprise any cationic surfactant.
Their zeta potentials are negative.
The complexing of siRNA did not occur on the surface of the droplets as was expected.
Formulation B9 is a comparative example since it does not comprise an amphiphilic lipid. It was not possible to prepare the emulsion.

The preparation method given below was followed:

(i) Preparation of the Oil Phase:

The soybean oil, suppocire NC, amphiphilic lipid, DOTAP, optional DOPE were weighed and mixed with dichloromethane before being heated to 60° C. to obtain a homogeneous viscous solution. The dichloromethane promotes solubilisation. The solvents were then evaporated in vacuo.

(ii) Preparation of the Aqueous Phase:

During the ethanol evaporation phase the aqueous phase was prepared. In a 5 ml Eppendorf tube, the co-surfactant, glycerol, and aqueous solution of PBS (154 mM NaCl, pH 7.4) were mixed then dissolved in a bath at 75° C.

(iii) Mixing of the Two Phases:

The oil phase was at about 40° C. (in viscous form) and the aqueous phase at about 70° C. (on leaving the bath). The aqueous bath was poured into the oil phase.

(iv) Emulsification:

The bottle containing the two phases was fitted inside the sonication chamber of an AV505® sonicator (Sonics, Newton, USA). The protocol entailed sonication cycles (10 seconds of activity every 30 seconds) at a power of 100 W over a period of 40 minutes.

(v) Purification:

The droplets produced were then purified by dialysis (cut-off threshold: 12 kDa, against 154 mM NaCl overnight) to remove the lipid components non-integrated in the LNPs. Finally the formulation was sterilised by filtration on a cellulose membrane.

Size of the Droplets of the Formulation and Zeta Potential

Influence of the Composition of the Formulation

The results in Tables 1 to 5 show that a decrease in the proportion of co-surfactant (Myrj S40) leads to an increase in the diameter of the droplets.

Trend in Time

The trend in droplet size (Table 6) and zeta potential (Table 7) of the formulations were measured at 40° C. (accelerated stability). The formulations were stored at 40° C. between two measurements.

TABLE 6
Trend in droplet size and polydispersity index (PDI) measured by
dynamic light scattering as a function of time

| Days | B1 | B4 | B5 | B6 | B10 | C1 | C2 |
|---|---|---|---|---|---|---|---|
| 0 | 58.27 | 98.87 | 60.03 | 87.93 | 91.53 | 157.43 | 171.4 |
| 7 | 60.52 | 97.12 | 58.96 | 87.1 | 90.51 | 156.17 | 172.63 |
| 14 | 62.59 | 98.36 | 58.28 | 86.25 | 89.87 | 156.46 | 170.15 |
| 21 | 59.41 | 97.81 | 59.23 | 85.78 | 90.3 | 153.03 | 162.2 |
| 28 | 61 | 97.15 | 60.36 | 87.61 | 90.96 | 153.9 | 161.47 |

TABLE 7
Trend in zeta potential of the formulations as a function of time

| Days | B1 | B4 | B5 | B6 | B10 | C1 | C2 |
|---|---|---|---|---|---|---|---|
| 0 | -21.3 | -21.16 | 24.03 | 34.13 | 34.97 | -40.27 | 57.33 |
| 7 | -25.6 | -21.17 | 28.23 | 31.77 | 34.73 | -43.13 | 56.77 |
| 14 | -24.73 | -21.03 | 29.45 | 28.4 | 33.47 | -42.33 | 55.33 |
| 21 | -21.8 | -20.47 | 28.63 | 34.3 | 33.07 | -38.1 | 53.9 |
| 28 | -18.93 | -19.83 | 27.5 | 33 | 31.23 | -39.03 | 51.6 |

It was observed that the size of the droplets and the zeta potential of the formulations according to the invention when stored at 40° C. for 300 days showed no change.

These results demonstrate that the formulations of the invention are stable over time.

Complexing with siRNA: Preparation of <<Final>> Formulations Comprising siRNA Nucleotide Sequences.

The general procedure below was followed:

Complexing involved simple mixing of the formulations prepared above and of a siRNA solution in a buffer. The choice of buffer related to the envisaged application: for an in vitro study, the optimised culture medium for the transfection steps was OptiMEM. For a complexing study the buffer used was 5 mM Hepes.

The amount of siRNA used was 0.5 μg (GFP-22 siRNA rhodamine (catalogue n^{o} 1022176) (Qiagen) or siGFP (Sigma)) (25 μg/mL in 20 μL).

The mixture was left under agitation for 30 minutes at 600 rpm, at ambient temperature (about 25° C.).

Complexing was visualised by means of two instruments:
Agarose gel electrophoresis which allowed observation of siRNA migration. If there is good complexing then the droplets comprising the complexed siRNA will be heavier than the free siRNA and will be seen in the wells. If complexing is less extensive, free siRNA migrate towards another position.
With DLS the impact of complexing on hydrodynamic diameter was observed. The more complexing is efficient the more the profile tends towards monomodal distribution.

The amount of formulation needed to obtain a quantitative complexing yield of siRNA was optimised.

In practice the negative charges provided by the siRNA are offset by the positive charges of the formulation (i.e. the positive charges of the cationic surfactant DOTAP).

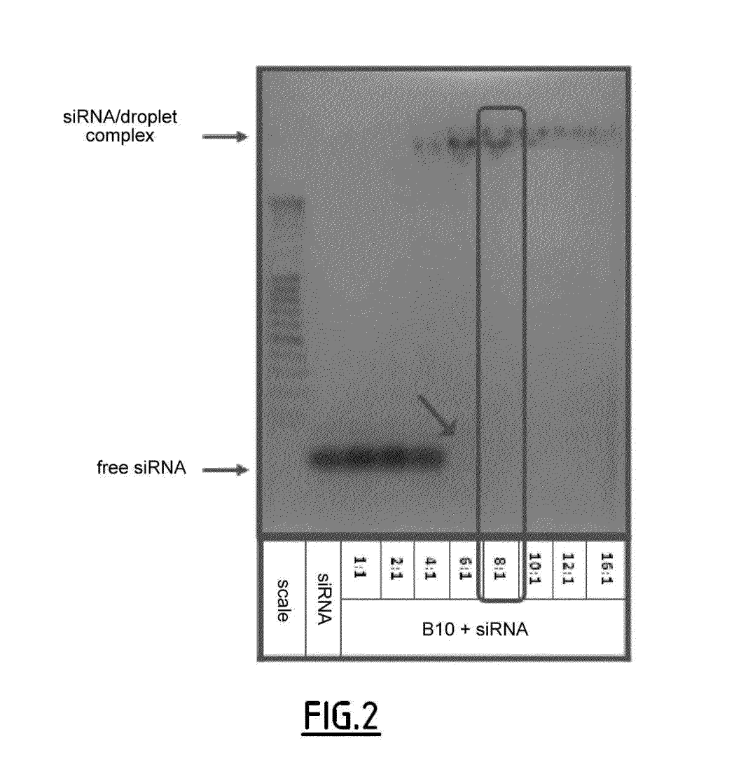

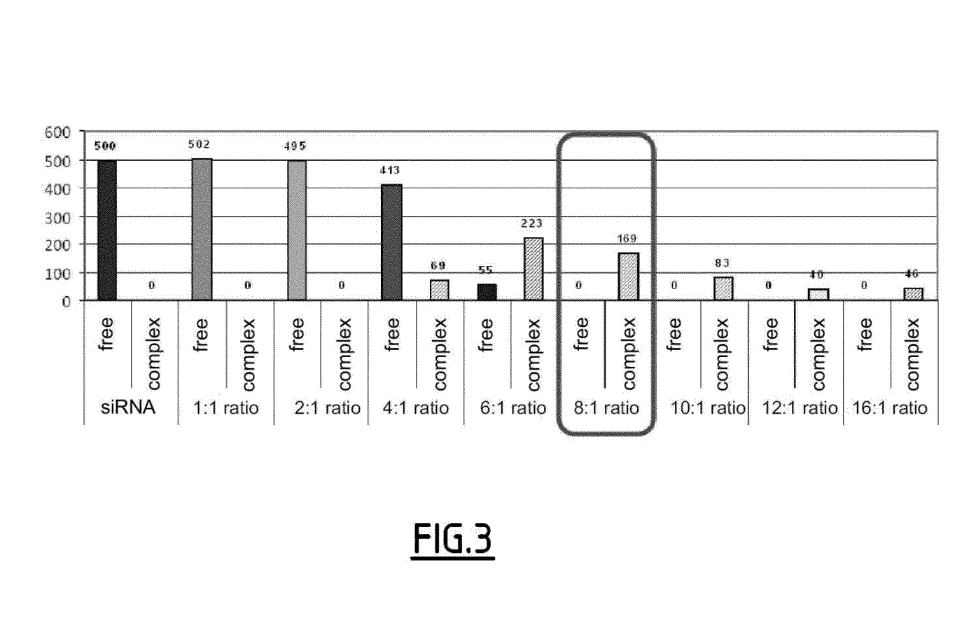

Typically, when the only cationic surfactant of the formulation is DOTAP (which only comprises a single positive charge) a quantitative complexing yield is obtained when the ratio of amount of positive charges due to the cationic surfactant in the <<premix>> formulation to the amount of negative charge provided by the siRNA is greater than 8:1 as illustrated in FIGS. 2 and 3.

FIG. 2 shows an electrophoresis gel with UV detection after complexing of siRNA with formulation B10 at the concentrations specified in Table 8 by mixing a solution of siRNA and the formulation in 5 mM Hepes buffer. Before depositing on 1.5% agarose gel, 2 μL of loading buffer were added to the tests. After 1 h 30 electrophoresis at 100 V, the gel was immersed in GelRed 3×. Finally UV detection was carried out.

TABLE 8

| | formulation | | | | | | | | |
|---|---|---|---|---|---|---|---|---|---|
| | and amount of | | | | | | | | |
| | negative | ratio between amount of positive | | | | | | | |
| | charges | charges due to the cationic | | | | | | | |
| | provided | surfactant in the [symbol] premix [symbol] | | | | | | | |
| | by siRNA | 1:1 | 2:1 | 4:1 | 6:1 | 8:1 | 10:1 | 12:1 | 16:1 |
| Concentration | | 25 | | | | | | | |
| of siARN | | | | | | | | | |
| (μg/mL) | | | | | | | | | |
| Concentration | 0 | 25 | 50 | 100 | 150 | 200 | 250 | 300 | 400 |
| of DOTAP | | | | | | | | | |
| (μg/mL) | | | | | | | | | |

FIG. 3 gives the results obtained by processing electrophoresis data on same experiments using ImageJ software.

FIGS. 2 and 3 show that when the ratio between the amount of positive charges due to the cationic surfactant in the <<premix>> formulation and the amount of negative charges provided by siRNA is greater than 8:1 there no longer remains any free siRNA in the medium and that the siRNA has been fully complexed.

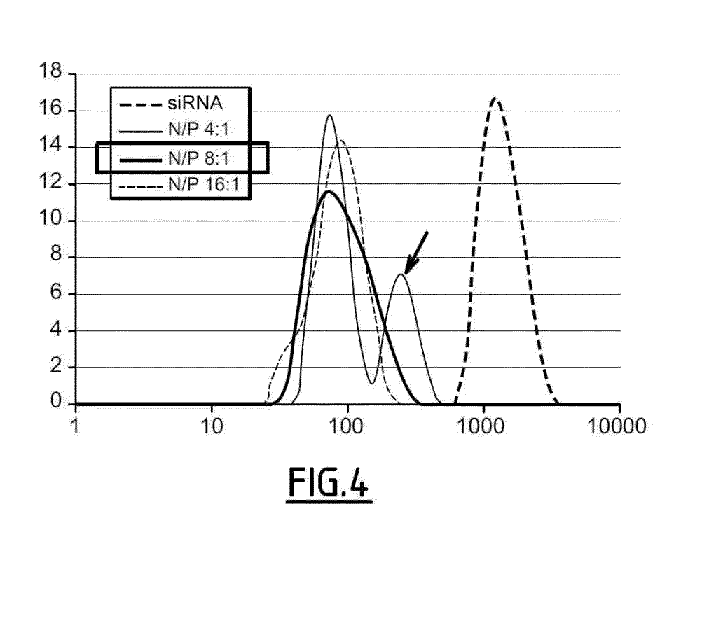

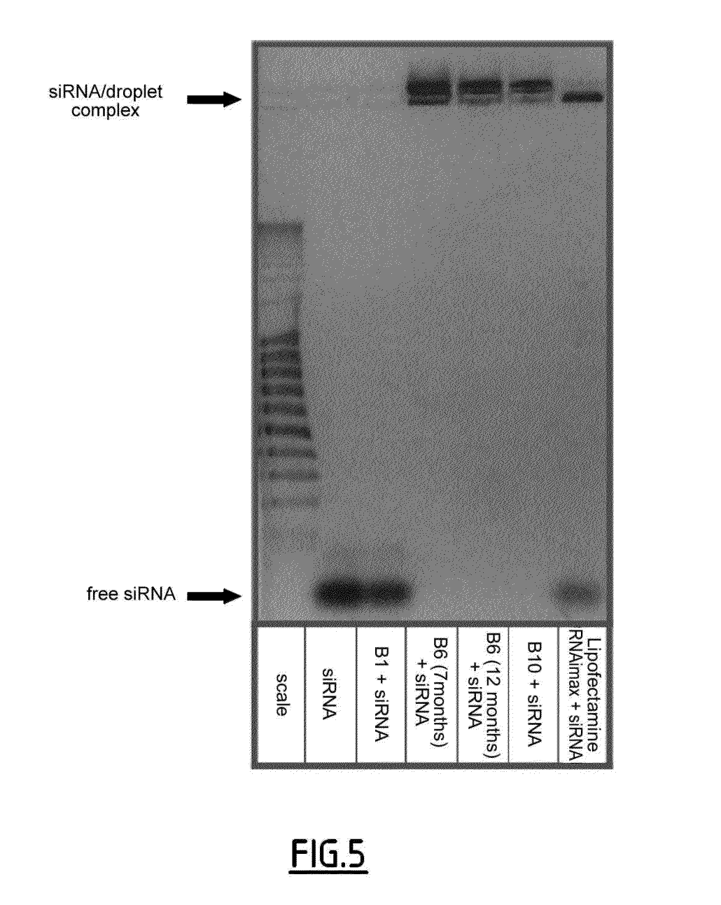

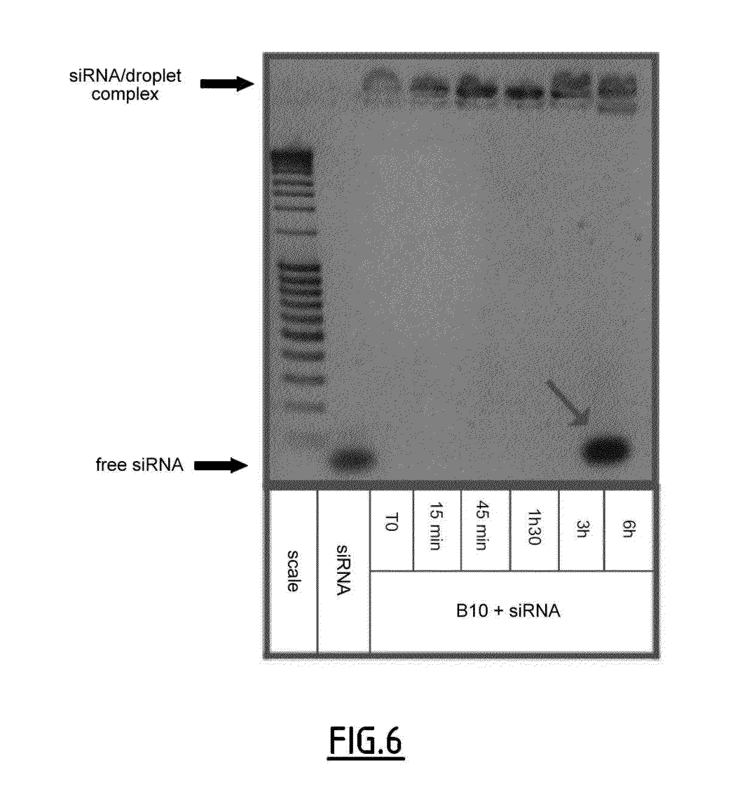

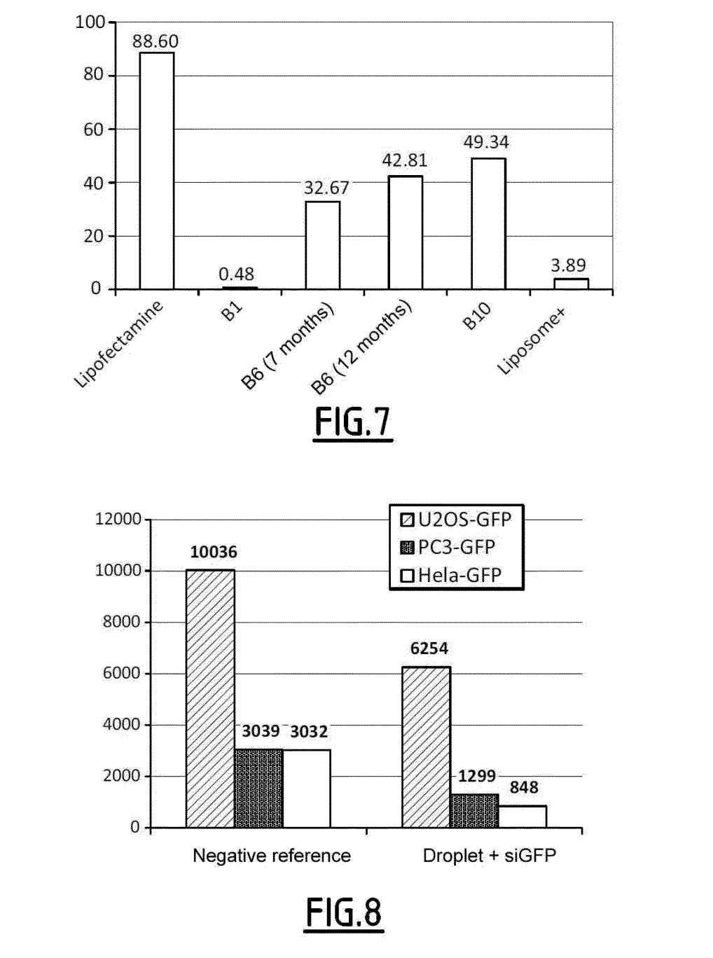

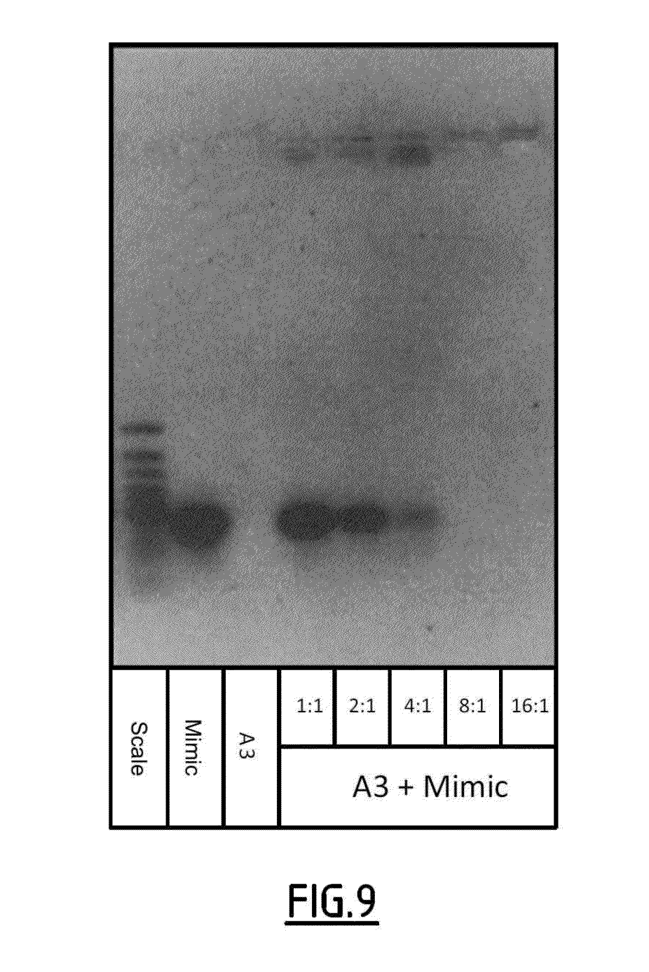

FIG. 4 shows the intensity obtained on Malvern ZetaSizer apparatus as a function of hydrodynamic diameter in nm for free siRNA (comparison) and for three formulations of the invention obtained by complexing with ratio values between amount of positive charges due to the cationic surfactant in the <<premix>> formulation and the amount of negative charges provided by siRNA of 4:1, 8:1 and 16:1.

The diameter was greater for free siRNA (comparison).

When the ratio between amount of positive charges due to the cationic surfactant in the <<premix>> formulation and amount of negative charges provided by siRNA is 4:1, two populations were observed: a siRNA/droplet complex of about 100 nm and a population of greater size representing siRNA in free form (arrow).

When the ratios of amount of positive charges due to the cationic surfactant in the <<premix>> formulation and amount of negative charges provided by the siRNA are 8:1 and 16:1, a single population representing the siRNA/droplet complex was observed.

These results also show that quantitative complexing (100%) of siRNA on the droplets is possible.

The complexing step was performed with various formulations.

By way of comparison, a complexing test was performed with a formulation free of cationic surfactant: formulation B1 described above. As expected, the complexing of the siRNA did not take place and the siRNA remained in free form.

FIG. 5 shows an electrophoresis gel with UV detection giving from left to right:
the scale,
free siRNA (reference)
   then the migrations obtained by siRNA complexing:
with formulation B1 (comparative example) (no complexing, the siRNA are free);
with formulation B6 (premix formulated 6 months previously) having a ratio between amount of positive charges due to the cationic surfactant in the <<premix>> formulation and amount of negative charges provided by the siRNA of 8:1 (quantitative complexing);
with formulation B6 (premix formulated 12 months previously) with a ratio between amount of positive charges due to the cationic surfactant in the <<premix>> formulation and amount of negative charges provided by the siRNA of 8:1, the formulation having been stored 7 months at ambient temperature prior to complexing (quantitative complexing);
with formulation B10 and a ratio between amount of positive charges due to the cationic surfactant in the <<premix>> formulation and amount of negative charges provided by the siRNA of 8:1 (quantitative complexing);
with lipofectamine (comparative example) (incomplete complexing).

The complexing of siRNA was quantitative when formulation B6 or B10 was used. The storage of the formulation at ambient temperature before complexing with siRNA did not have any influence on the yield of complexing which remained quantitative, thereby showing the stability of the formulations used.

The yield was quantitative whether or not the formulation used contained DOPE.

With the commercial transfection agent Lipofectamine RNAimax, more than 60% of the siRNA were found in free form. This implies that the formulations of nanoemulsions used in the invention offer a better complexing yield than Lipofectamine.

Finally the release kinetics of siRNA in the siRNA/formulation B10 complex prepared above were studied to observe the trend in complexing over time and are illustrated in FIG. 6. Up to 3 hours after complexing the siRNA are not found in free form. At 6 hours the siRNA start to be released. The complex is therefore stable for at least three hours.

In Vitro Transfection

Transfection tests were conducted on different cell lines overexpressing Green Fluorescent Protein (GFP) by providing a siRNA specifically inhibiting a sequence of messenger RNA of this protein (GFP-22 siRNA rhodamine (catalog n^{o} 1022176) (Qiagen)).

Transfection was performed with a final siRNA concentration of 100 nM. Cells expressing GFP were seeded in 12-well plates (25 000 cells/well) and the wells were treated with the complexes siRNA/Formulations (B1, B6, B6 7 months after its preparation or B10) obtained above. The cells were then incubated 72 hours at 37° C., and recovered for analysis of fluorescence intensity by flow cytometry to determine the efficacy of the formulation as transfecting agent. The active delivery of siRNA specifically inhibiting the expression of the GFP protein causes a decrease in the fluorescence provided by this protein.

The commercial transfecting agent Lipofectamine RNAimax was used for comparison.

FIG. 7 shows the % decrease in fluorescence intensity when transfecting the cell lines with:
Lipofectamine RNAimax;
the siRNA/formulation B1 complex;
the siRNA/formulation B6 complex, the formulation having been stored 7 months at ambient temperature prior to complexing;
the siRNA/formulation B6 complex, the formulation having been stored 12 months at ambient temperature prior to complexing;
the siRNA/formulation B10 complex,
a complex siRNA/formulation of cationic liposomes comprising DOTAP (58 wt. %), DOPE (18 wt. %), cholesterol (2 wt. %) and DSPE-PEG3000 (22 wt. %)) (comparative).

A decrease in fluorescence of 33 to 50% is observed with the tested formulations of the invention. The formulations of the invention therefore allow active delivery of siRNA inducing relative silencing of the expression of the GFP gene.

In addition, by incorporating DOPE in the formulations, a greater decrease in fluorescence was observed, DOPE promoting endosomal escape.

No decrease in fluorescence was observed with the complex of siRNA./formulation of cationic liposomes which could be attributed for example to poor stability of the liposomes in the culture medium, poor complexing yield and/or poor retaining of siRNA by the liposomes after complexing.

Finally such results on the active delivery of siRNA mediated by the formulations of the invention were reproduced on 3 cell lines expressing GFP: U2OS, PC3 and Hela, as illustrated in FIG. 8.

Complexing with Synthetic microRNA: Preparation of <<Final>> Formulations Comprising Nucleotide Sequences of Synthetic microRNA (Mimic).

The general procedure given below was followed:

Complexing involved simple mixing of the A3 premix formulation prepared above with a solution of microRNA (miRIDIAN Mimic Human has-miR612 (ThermoScientific REF#C-300937-01 Batch n^{o} 130611)), in a buffer. The choice of buffer was dependent on the envisaged application: for an in vitro study the optimised culture for transfection steps OptiMEM®, was used. For a study on complexing 5 mM Hepes buffer was used.

An amount of 0.5 μg of microRNA was used (miRIDIAN Mimic Human has-miR612 (ThermoScientific REF#C-300937-01 Batch n^{o} 130611).

The mixture was left under agitation 30 minutes at 600 rpm, at ambient temperature (about 25° C.).

Complexing was visualised by detection on agarose gel obtained by electrophoresis which allowed observation of microRNA migration. With good complexing the droplets comprising the complexed microRNA are heavier than the free microRNA and can be seen in the wells. If complexing is less extensive, free microRNA migrate towards another position.

The amount of A3 premix formulation required to obtain a quantitative yield of microRNA was optimised.

In practice the negative charges provided by the microRNA are offset by the positive charges of the premix formulation (i.e. the positive charges of the cationic surfactant DOTAP). Typically, when the sole cationic surfactant of the premix formulation is DOTAP (which only comprises a single positive charge) a quantitative yield of complexing is obtained when the ratio between amount of positive charges due to the cationic surfactant in the <<premix>> formulation and amount of negative charges provided by the microRNA (N/P ratio) is greater than 8:1, as previously with the siRNA.

FIG. 9 shows an electrophoresis gel with UV detection after complexing microRNA with formulation A3 at the concentrations specified in Table 9, by mixing a solution of microRNA and the A3 premix formulation in 5 mM HEPES buffer. Before depositing on 1.5% agarose gel, 2 μL of loading buffer were added to the tests. After 1 h 30 electrophoresis at 100 V, the gel was immersed in GelRed 3×. Finally UV detection was carried out.

TABLE 9

| | formulation | | | | | | | | |
|---|---|---|---|---|---|---|---|---|---|
| | and amount of | | | | | | | | |
| | negative | ratio between amount of positive | | | | | | | |
| | charges | charges due to the cationic surfactant in | | | | | | | |
| | provided by | the [symbol] premix [symbol] | | | | | | | |
| | microRNA | 1:1 | 2:1 | 4:1 | 6:1 | 8:1 | 10 1 | 12:1 | 16:1 |
| Concentration | 25 | | | | | | | | |
| of microRNA | | | | | | | | | |
| (μg/mL) | | | | | | | | | |
| Concentration | 0 | 25 | 50 | 100 | 150 | 200 | 250 | 300 | 400 |
| of DOTAP | | | | | | | | | |
| (μg/mL) | | | | | | | | | |

FIG. 9 shows that for a ratio value between amount of positive charges due to the cationic surfactant in the <<premix>> formulation and amount of negative charges provided by microRNA higher than 8:1, there are no longer any free microRNA remaining in the medium and that the microRNA has been fully complexed, as illustrated in FIGS. 2 and 3 with siRNA.

1. A formulation in nanoemulsion form comprising a continuous aqueous phase and a least one dispersed phase, and comprising:
at least 5 mole % of amphiphilic lipid;
15 to 70 mole % of at least one cationic surfactant comprising:
at least one lipophilic group selected from among:
an R or R—(C═O)— group, where R is a linear hydrocarbon chain having 11 to 23 carbon atoms,
an ester or amide of fatty acids having 12 to 24 carbon atoms and phosphatidylethanolamine, and
a poly(propylene oxide), and
at least one hydrophilic group comprising at least one cationic group selected from among:
a linear or branched alkyl group having 1 to 12 carbon atoms and interrupted and/or substituted by at least one cationic group; and
a hydrophilic polymeric group comprising at least one cationic group; and
10% to 55 mole % of a co-surfactant comprising at least one poly(ethylene oxide) chain comprising at least 25 ethylene oxide units;
a solubilising lipid,
optionally a helper lipid,
where the mole percentages of amphiphilic lipid, cationic surfactant and co-surfactant are relative to the whole (amphiphilic lipid/cationic surfactant/co-surfactant/optional helper lipid).

2. The formulation according to claim 1 wherein the cationic surfactant is selected from among:
N[1-(2,3-dioléyloxyl)propyl]-N,N,N-trimethylammonium,
1,2-dioleyl-3-trimethylamonium-propane,
N-(2-hydroxyethyl)-N,N-dimethyl-2,3-bis(tetradecyloxy-1-propananium),
1-[2-(oleoyloxy)ethyl]-2-oleyl-3-(2-hydroxyethyl)imidazolinium, and
dioctadecylamidoglycylspermine.

3. The formulation according to claim 1 comprising a helper lipid.

4. The formulation according to claim 1 wherein the amphiphilic lipid is a phospholipid.

5. The formulation according to claim 1 comprising an imaging agent.

6. The formulation according to claim 1 comprising a therapeutic agent.

7. The formulation according to claim 1 comprising a co-surfactant grafted with a molecule of interest.

8. The formulation according to claim 1 comprising a single or double strand nucleotide sequence, comprising fewer than 200 bases for a single strand nucleotide sequences or fewer than 200 base pairs for a double strand nucleotide sequence, able to modulate endogenous mechanisms of RNA interference.

9. The formulation according to claim 8 wherein the said nucleotide sequence is selected from among:
small interfering RNA;
locked nucleic acid; and
synthetic microRNA.

10. The formulation according to claim 9 wherein the said nucleotide sequence is small interfering RNA.

11. A method for preparing a formulation according to claim 1, wherein the formulation comprises a single or double strand nucleotide sequence, comprising fewer than 200 bases for a single strand nucleotide sequences or fewer than 200 base pairs for a double strand nucleotide sequence, able to modulate endogenous mechanisms of RNA interference, the method comprising the following steps:
(i) preparing an oil phase comprising a solubilising lipid, an amphiphilic lipid, the cationic surfactant;
(ii) preparing an aqueous phase comprising the co-surfactant;
(iii) dispersing the oil phase in the aqueous phase under sufficient shear action to obtain a formulation in nanoemulsion form such as defined in claim 1; then
(iv) adding a single or double strand nucleotide sequence, comprising fewer than 200 bases for a single strand nucleotide sequence or fewer than 200 base pairs for a double strand nucleotide sequence, able to modulate endogenous mechanisms of RNA interference, to the formulation in emulsion form such as defined in claim 1, then
(v) recovering the formulation thus formed.

12. A method for in vitro insertion into a eukaryote cell of a single or double strand nucleotide sequence, comprising fewer than 200 bases for a single nucleotide sequence or fewer than 200 base pairs for a double strand nucleotide sequence, able to modulate endogenous mechanisms of RNA interference, comprising the contacting of the eukaryote cell with a formulation according to claim 8.

13. A method for the prevention and/or treatment of a disease comprising the administration to a mammal in need thereof, of an efficient Quantity of the formulation according to claim 8 for use thereof to prevent and/or treat a disease.

14. A kit comprising the formulation in nanoemulsion form as defined in claim 1 and, separately, at least one single or double strand nucleotide sequence comprising fewer than 200 bases for a single strand nucleotide sequence or fewer than 200 base pairs for a double strand nucleotide sequence, that is able to modulate endogenous mechanisms of RNA interference.

15. The formulation according to claim 3, wherein the helper lipid is 1,2-Dioleoyl-sn-Glycero-3-Phosphoethanolamine.

16. The formulation according to claim 7, wherein the molecule of interest, is a targeting biological ligand.
